SEQUENCE LISTING (52 sequences)

SEQ ID NO: 1 - DNA, 7320 bp, Zea mays
tgatgctttttcctgggcagggagagctatgagacgtatgtcctcaaagccactttgcattgtgtgaaaccaatatcgatctttgttacttcatcatgcatgaacatttgtggaaactactagcttacaagcattagtgacagctcagaaaaaagttatctctgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctattcacctatctctcgtgcctgtttattttcttgccctttctgatcataaaaaatcattaagagtttgcaaacatgcataggcatatcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaatcggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctcctcatactctatataaacacatcaacagcatcgcaattagccacaagatcacttcgggaggcaagtgtgatttcgaccttgcagccacctttttttgttctgttgtaagtatactttcccttaccatctttatctgttagtttaatttgtaattgggaagtattagtggaaagaggatgagatgctatcatctatgtactctgcaaatgcatctgacgttatatgggctgcttcatataatttgaattgctccattcttgccgacaatatattgcaaggtatatgcctagttccatcaaaagttctgttttttcattctaaaagcattttagtggcacgcaattttgtccatgagggaaaggaaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttgtaagtatccacctcaattattactcttacatgttggtttactttacgtttgtcttttcaagggaaatttactgtattttttgtgttttgtgggagttctatacttctgttggactggttattgtaaagatttgttcaaatagggtcatctaataattgtttgaaatctgggaactgtggtttcactgcgttcaggaaaaagtgaattattggttactgcatgaataacttatggaaatagaccttagagttgctgcatgattatcacaaatcattgctacgatatcttataatagttctttcgacctcgcattacatatataactgcaactcctagttgcgttcaaaaaaaaaaatgcaactcttagaacgctcaccagtgtaatctttcctgaattgttatttaatggcatgtatgcactacttgtatacttatctaggattaagtaatctaactctaggccccatatttgcagcattctcaaacacagtcctctaggaaaaattatgctgatgcaaaccgtgtatctgctatcattttgggcggaggcactggatctcagctctttcctctgacaagcacaagagctacgcctgctgtaagggataacactgaacatccaacgttgattactctattatagtattatacagactgtacttttcgaatttatcttagttttctacaatatttagtggattcttctcattttcaagatacacaattgatccataatcgaagtggtatgtaagacagtgagttaaaagattatattttttgggagacttccagtcaaattttcttagaagtttttttggtccagatgttcataaagtcgccgctttcatactttttttaattttttaattggtgcactattaggtacctgttggaggatgttacaggcttattgatatccctatgagtaactgcttcaacagtggtataaataagatatttgtgatgagtcagttcaattctacttcgcttaaccgccatattcatcgtacataccttgaaggcgggatcaactttgctgatggatctgtacaggtgatttacctcatcttgttgatgtgtaatactgtaattaggagtagatttgtgtggagagaataataaacagatgccgagattcttttctaaaagtctagatccaaaggcattgtggttcaaaacactatggacttctaccatttatgtcattactttgccttaatgttccattgaatggggcaaattattgattctacaagtgtttaattaaaaactaattgttcatcctgcaggtattagcggctacacaaatgcctgaagagccagctggatggttccagggtacagcagactctatcagaaaatttatctgggtactcgaggtagttgatattttctcgtttatgaatgtccattcactcattcctgtagcattgtttctttgtaattttgagttctcctgtatttctttaggattattacagtcacaaatccattgacaacattgtaatcttgagtggcgatcagctttatcggatgaattacatggaacttgtgcaggtatggtgttctcttgttcctcatgtttcacgtaatgtcctgattttggattaaccaactacttttggcatgcattatttccagaaacatgtcgaggacgatgctgatatcactatatcatgtgctcctgttgatgagaggtaatcagttgtttatatcatcctaatatgaatatgtcatcttgttatccaacacaggatgcatatggtctaatctgctttccttttttttcccttcggaagccgagcttctaaaaatgggctagtgaagattgatcatactggacgtgtacttcaattctttgaaaaaccaaagggtgctgatttgaattctatggttagaaattccttgtgtaatccaattcttttgttttcctttctttcttgagatgaacccctcttttagttatttccatggataacctgtacttgacttattcagaaatgattttctattttgctgtagaatctgacactaaagctaatagcactgatgttgcagagagttgagaccaacttcctgagctatgctatagatgatgcacagaaatatccataccttgcatcaatgggcatttatgtcttcaagaaagatgcacttttagaccttctcaagtaatcactttcctgtgacttatttctatccaactcctagtttaccttctaacagtgtcaattcttaggtcaaaatatactcaattacatgactttggatctgaaatcctcccaagagctgtactagatcatagtgtgcaggtaagtctgatctgtctggagtatgtgttctgtaaactgtaaattcttcatgtcaaaaagttgtttttgtttccagtttccactaccaatgcacgatttatgtattttcgcttccatgcatcatacatactaacaatacattttacgtattgtgttaggcatgcatttttacgggctattgggaggatgttggaacaatcaaatcattctttgatgcaaacttggccctcactgagcaggtactctgtcatgtattctgtactgcatatatattacctggaattcaatgcatagaatgtgttagaccatcttagttccatcctgttttcttcaattagcttatcatttaatagttgttggctagaatttaaacacaaatttacctaatatgtttctctcttcagccttccaagtttgatttttacgatccaaaaacacctttcttcactgcaccccgatgcttgcctccgacgcaattggacaagtgcaaggtatatgtcttactgagcacaattgttacctgagcaagattttgtgtacttgacttgttctcctccacagatgaaatatgcatttatctcagatggttgcttactgagagaatgcaacatcgagcattctgtgattggagtctgctcacgtgtcagctctggatgtgaactcaaggtacatactctgccaatgtatctactcttgagtataccatttcaacaccaagcatcaccaaatcacacagaacaatagcaacaaagccttttagttccaagcaatttagggtagcctagagttgaaatctaacaaaacaaaagtcaaagctctatcacgtggatagttgttttccatgcactcttatttaagctaattttttgggtatactacatccatttaattattgttttattgcttcttccctttgcctttcccccattactatcgcgtcttaagatcatactacgcactagtgtctttagaggtctctggtggacatgttcaaaccatctcaatcggtgttggacaagtttttcttgaatttgtgctacacctaacctatcacgtatgtcatcgtttcaaactcgatccttcctgtatcatcataaatccaatgcaacatacgcatttatgcaacatttatctgttgaacatgtcatctttttgtaggttaacattatgcaccatacaatgtagcatgtctaatcatcatcctataaaatttacattttagcttatgtggtatcctcttgccacttagaacaccatatgcttgatgccatttcatccaccctgctttgattctatggctaacatcttcattaatatcctcgcctctctgtatcattggtcctaaatatggaaatacattctttctgggcactacttgaccttccaaactaacgtctcctttgctcctttcttgtgtgtagtagtaccgaagtcacatctcatatattcggttttagttctactaagtcccgggttcgatccccctcaggggtgaatttcgggcttggtaaaaaaaatcccctcgctgtgtcccgcccgctctcggggatcgatatcctgcgcgccaccctccggctgggcattgcagagtgagcagttgatcggctcgttagtgatggggagcggggttcaagggttttctcggccgggaccatgtttcggtctcttaatataatgccgggagggcagtctttccctccccggtcgagttttagttctaccgagtctaaaacctttggactctagagtcccctgtcacaactcacaactctagttttctatttacttctacctagcgtttattaatgatcactatatcgtctgtaaaaagcatacaccaatgtaatccccttgtatgtcccttgtaatattatccatcacaagaaaaaaaggtaaggctcaaagttgacttttgatatagtcctattctaatcgagaagtcatctgtatcttcgtctcttgttcgaacactagtcacaaaattttttgtacatgttcttaatgagtccaacgtaatattccttgatattttgtcataagccctcatcaagtcaatgaaaatcacgtgtaggtccttcatttgttccttatactgctccatcacttgtctcattaagaaaatctctctcatagttaaccttttggcatgaaacaaaatcacacagaagttgtttcctttttttaagatcccacacaaaagaggtttgatctaaggaatctggatccctgacaggtttatcaaaatcctttgtgtttttcttaaaactgaatattcctccagcttctagtattgatgtaatattcaatctgtttagcaagtgaacaccttggttcttgttgttactgtaccccccccccccccccccccccgaggcccagattaccacgacatgaatacaagaatattgaacccagatctagagtttgtttgtactgttgaaaatcggtgacaattcattttgttattgcgctttctgataacgacaggactccgtgatgatgggagcggacacctatgaaactgaagaagaagcttcaaagctactgttagctgggaaggtcccagttggaataggaaggaacacaaagataaggtgagtatggatgtggaaccaccggttagttcccaaaaatatcactcactgatacctgatggtatcctctgattattttcaggaactgtatcattgacatgaatgctaggattgggaagaacgtggtgatcacaaacagtaaggtgagcgagcgcacctacatgggtgcagaatcttgtgtgctcatctatcctaattcggtaattcctatccagcgctagtcttgtgaccatggggcatgggttcgactctgtgacagggcatccaagaggctgatcacccggaagaagggtactacataaggtctggaatcgtggtgatcttgaagaatgcaaccatcaacgatgggtctgtcatatagatcggctgcgtgtgcgtctacaaaacaagaacctacaatggtattgcatcgatggatcgtgtaaccttggtatggtaagagccgcttgacagaaagtcgagcgttcgggcaagatgcgtagtctggcatgctgttccttgaccatttgtgctgctagtatgtactgttataagctgccctagaagttgcagcaaacctttttatgaacctttgtatttccattacctgctttggatcaactatatctgtcatcctatatattactaaatttttacgtgtttttctaattcggtgctgcttttgggat

SEQ ID NO: 2 - DNA, 81 bp, Zea mays
  variation (41): Base G
ttgtgtgaaaccaatatcgatctttgttacttcatcatgcatgaacatttgtggaaactactagcttacaagcattagtga

SEQ ID NO: 3 - DNA, 81 bp, Zea mays
  variation (41): Base G
aatttgctagcagatcatcttcctactctttactttatttattgtttgaaaaatatgtcctgcacctagggagctcgtata

SEQ ID NO: 4 - DNA, 80 bp, Zea mays
  variation (41): Base A
gtatacagtaccaatgcatcttcattaaatgtgaatttcagaaaggaagtaggaacctatgagagtatttttcaaaatta

SEQ ID NO: 5 - DNA, 81 bp, Zea mays
  variation (41): Base T
gcatcttcattaaatgtgaatttcagaaaggaagtaggaacctatgagagtatttttcaaaattaattagcggcttctatt

SEQ ID NO: 6 - DNA, 81 bp, Zea mays
  variation (41): Base T
atttttcaaaattaattagcggcttctattatgtttatagcaaaggccaagggcaaaatcggaacactaatgatggttggt

SEQ ID NO: 7 - DNA, 81 bp, Zea mays
  variation (41): Base T
cggcttctattatgtttatagcaaaggccaagggcaaaatcggaacactaatgatggttggttgcatgagtctgtcgatta

SEQ ID NO: 8 - DNA, 81 bp, Zea mays
  variation (41): Base C
atgtttatagcaaaggccaagggcaaaatcggaacactaatgatggttggttgcatgagtctgtcgattacttgcaagaaa

SEQ ID NO: 9 - DNA, 81 bp, Zea mays
  variation (41): Base A
gtgcgtgggcataaaacaaacagcttctagcctcttttacggtacttgcacttgcaagaaatgtgaactccttttcatttc

SEQ ID NO: 10 - DNA, 81 bp, Zea mays
  variation (41): Base A
ccagtatgccctcctcatactctatataaacacatcaacagcatcgcaattagccacaagatcacttcgggaggcaagtgt

SEQ ID NO: 11 - DNA, 81 bp, Zea mays
  variation (41): Base T
ttagccacaagatcacttcgggaggcaagtgtgatttcgaccttgcagccacctttttttgttctgttgtaagtatacttt

SEQ ID NO: 12 - DNA, 81 bp, Zea mays
  variation (41): Base C
tgcatactcatttatttgagttgcaggtgtatctagtagttggaggagatatgcagtttgcacttgcattggacacgaact

SEQ ID NO: 13 - DNA, 81 bp, Zea mays
  variation (41): Base C
acacgaactcaggtcctcaccagataagatcttgtgagggtgatgggattgacaggttggaaaaattaagtattgggggca

SEQ ID NO: 14 - DNA, 81 bp, Zea mays
  variation (41): Base A
gaaagctttgagaaataggtgctttggtggtagagttgctgcaactacacaatgtattcttacctcagatgcttgtcctga

SEQ ID NO: 15 - DNA, 81 bp, Zea mays
  variation (41): Base A
ggaaaaagtgaattattggttactgcatgaataacttatggaaatagaccttagagttgctgcatgattatcacaaatcat

SEQ ID NO: 16 - DNA, 81 bp, Zea mays
  variation (41): Base G
tagttctttcgacctcgcattacatatataactgcaactcctagttgcgttcaaaaaaaaaaatgcaactcttagaacgct

SEQ ID NO: 17 - DNA, 81 bp, Zea mays
  variation (41): Base G
cgacctcgcattacatatataactgcaactcctagttgcgttcaaaaaaaaaaatgcaactcttagaacgctcaccagtgt

SEQ ID NO: 18 - DNA, 81 bp, Zea mays
  variation (41): Base C
cattttcaagatacacaattgatccataatcgaagtggtatgtaagacagtgagttaaaagattatattttttgggagact

SEQ ID NO: 19 - DNA, 81 bp, Zea mays
  variation (41): Base C
atttgtgtggagagaataataaacagatgccgagattcttttctaaaagtctagatccaaaggcattgtggttcaaaacac

SEQ ID NO: 20 - DNA, 81 bp, Zea mays
  variation (41): Base G
taatcactttcctgtgacttatttctatccaactcctagtttaccttctaacagtgtcaattcttaggtcaaaatatactc

SEQ ID NO: 21 - DNA, 81 bp, Zea mays
  variation (41): Base C
cttctaacagtgtcaattcttaggtcaaaatatactcaattacatgactttggatctgaaatcctcccaagagctgtacta

SEQ ID NO: 22 - DNA, 87 bp, Zea mays
aaaagttatctctgaaaggtttcatgtgtaccgtgggaaatgagaaatgttgccaactcaaacaccttcaatatgttgtttgcaggc

SEQ ID NO: 23 - DNA, 80 bp, Zea mays
aaaagttatctctgaaaggtttcatgtgtaccgtgggaaatgttgccaactcaaacaccttcaatatgttgtttgcaggc

SEQ ID NO: 24 - DNA, 88 bp, Zea mays
tgtctgctttatgatggcatctccacccaggcacccaccatcacctattcacctatctctcgtgcctgtttattttcttgccctttct

SEQ ID NO: 25 - DNA, 80 bp, Zea mays
tgtctgctttatgatggcatctccacccaggcacccaccatcacctatctctcgtgcctgtttattttcttgccctttct

SEQ ID NO: 26 - DNA, 80 bp, Zea mays
gttttttcattctaaaagcattttagtggcacgcaattttgtccatgagggaaaggaaatctgttttggttactttgctt

SEQ ID NO: 27 - DNA, 81 bp, Zea mays
gttttttcattctaaaagcattttagtggcacgcaatttttgtccatgagggaaaggaaatctgttttggttactttgctt

SEQ ID NO: 28 - DNA, 81 bp, Zea mays
catatataactgcaactcctagttgcgttcaaaaaaaaaaatgcaactcttagaacgctcaccagtgtaatctttcctgaa

SEQ ID NO: 29 - DNA, 80 bp, Zea mays
catatataactgcaactcctagttgcgttcaaaaaaaaaatgcaactcttagaacgctcaccagtgtaatctttcctgaa

SEQ ID NO: 30 - DNA, 80 bp, Zea mays
agtggtatgtaagacagtgagttaaaagattatattttttgggagacttccagtcaaattttcttagaagtttttttggt

SEQ ID NO: 31 - DNA, 81 bp, Zea mays
agtggtatgtaagacagtgagttaaaagattatatttttttgggagacttccagtcaaattttcttagaagtttttttggt

SEQ ID NO: 32 - DNA, 80 bp, Zea mays
tttgggagacttccagtcaaattttcttagaagtttttttggtccagatgttcataaagtcgccgctttcatactttttt

SEQ ID NO: 33 - DNA, 81 bp, Zea mays
tttgggagacttccagtcaaattttcttagaagttttttttggtccagatgttcataaagtcgccgctttcatactttttt

SEQ ID NO: 34 - DNA, 81 bp, Zea mays
aacacaggatgcatatggtctaatctgctttccttttttttcccttcggaagccgagcttctaaaaatgggctagtgaaga

SEQ ID NO: 35 - DNA, 80 bp, Zea mays
aacacaggatgcatatggtctaatctgctttcctttttttcccttcggaagccgagcttctaaaaatgggctagtgaaga

SEQ ID NO: 36 - DNA, 80 bp, Zea mays
tttctattttgctgtagaatctgacactaaagctaatagcactgatgttgcagagagttgagaccaacttcctgagctat

SEQ ID NO: 37 - DNA, 81 bp, Zea mays
tttctattttgctgtagaatctgacactaaagctaatagctactgatgttgcagagagttgagaccaacttcctgagctat

SEQ ID NO: 38 - DNA, 80 bp, Zea mays
tcttcatgtcaaaaagttgtttttgtttccagtttccactaccaatgcacgatttatgtattttcgcttccatgcatcat

SEQ ID NO: 39 - DNA, 91 bp, Zea mays
tcttcatgtcaaaaagttgtttttgtttccagtttccactgtttttatttaaccaatgcacgatttatgtattttcgcttccatgcatcat

SEQ ID NO: 40 - DNA, 18 bp, Artificial sequence
  Description of the artificial sequence  primer
ctgggcagggagagctat

SEQ ID NO: 41 - DNA, 23 bp, Artificial sequence
  Description of the artificial sequence  primer
ggatatcaataagcctgtaacat

SEQ ID NO: 42 - DNA, 20 bp, Artificial sequence
  Description of the artificial sequence  primer
tgcagcattctcaaacacag

SEQ ID NO: 43 - DNA, 22 bp, Artificial sequence
  Description of the artificial sequence  primer
cgatgttgcattctctcagtaa

SEQ ID NO: 44 - DNA, 20 bp, Artificial sequence
  Description of the artificial sequence  primer
atgtcctgcacctagggagc

SEQ ID NO: 45 - DNA, 20 bp, Artificial sequence
  Description of the artificial sequence  primer
ccaccagtatgccctcctca

SEQ ID NO: 46 - DNA, 21 bp, Artificial sequence
  Description of the artificial sequence  primer
gaccctatttgaacaaatctt

SEQ ID NO: 47 - DNA, 22 bp, Artificial sequence
  Description of the artificial sequence  primer
gcagcaactctaaggtctattt

SEQ ID NO: 48 - DNA, 20 bp, Artificial sequence
  Description of the artificial sequence  primer
tttgggagacttccagtcaa

SEQ ID NO: 49 - DNA, 19 bp, Artificial sequence
  Description of the artificial sequence  primer
catcgtcctcgacatgttt

SEQ ID NO: 50 - DNA, 21 bp, Artificial sequence
  Description of the artificial sequence  primer
ggaaagcagattagaccatat

SEQ ID NO: 51 - DNA, 23 bp, Artificial sequence
  Description of the artificial sequence  primer
tggaaactggaaacaaaaacaac

SEQ ID NO: 52 - protein, 516 aa, Zea mays
MQFALALDTNSGPHQIRSCEGDGIDRLEKLSIGGRKQEKALRNRCFGGRVAATTQCILTSDACPETLHSQTQSSRKNYADANRVSAIILGGGTGSQLFPLTSTRATPAVPVGGCYRLIDIPMSNCFNSGINKIFVMSQFNSTSLNRHIHRTYLEGGINFADGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYTQLHDFGSEILPRAVLDHSVQACIFTGYWEDVGTIKSFFDANLALTEQPSKFDFYDPKTPFFTAPRCLPPTQLDKCKMKYAFISDGCLLRECNIEHSVIGVCSRVSSGCELKDSVMMGADTYETEEEASKLLLAGKVPVGIGRNTKIRNCIIDMNARIGKNVVITNSKGIQEADHPEEGYYIRSGIVVILKNATINDGSVI

(19) United States
(12) Patent Application Publication
(10) Pub. No.: US 2005/0250205 A1
(43) Pub. Date: Nov. 10, 2005

(54) Use of associations between at least one nucleic sequence polymorphism of the sh2 gene and at least one seed quality characteristic in plant selection methods

(76) Inventors: Domenica Manicacci, Saint Aubin (FR); Dominique De Vienne, Fontenay Aux Roses (FR); Mathilde Causse, Le Thor (FR); Jean-Louis Prioul, Orsay (FR); Pascal Touzet, Bersee (FR); Matthieu Falque, Gif-Sur-Yvette (FR)

Correspondence Address: JACOBSON HOLMAN PLLC, 400 SEVENTH STREET N.W., SUITE 600, WASHINGTON, DC, 20004, US

(21) Appl. No.: 10/486,968

(22) PCT Filed: Aug. 16, 2002
(86) PCT No.: PCT/FR02/02898

§ 371 Date: Feb. 17, 2004

(30) Foreign Application Priority Data
Aug. 17, 2001 (FR) 01/10906

(51) Int. Cl. C12N 15/82; C12N 15/29; A01H 1/00

(52) U.S. Cl. 435/419; 435/6; 536/24.3; 536/23.1; 435/320.1; 435/468; 800/278; 800/298; 530/386

(57) ABSTRACT

The invention relates to the use of a nucleotide probe or of a nucleotide primer in a process for selecting plants having improved phenotypic seed quality characteristics, for detecting a polymorphic base or a polymorphic nucleotide sequence defining an allele of a polymorphic site of the Sh2 gene of sequence SEQ ID No. 1, said polymorphic base or said polymorphic nucleotide sequence being contained in a nucleic acid included in an Sh2 gene. Application to the production of transformed plants capable of producing seeds with improved industrial or agrofoods qualities.

### FIELD OF THE INVENTION

The present invention relates to the field of the selection of varieties of plants having improved agronomic characteristics, in particular improved phenotypic seed quality characteristics. It relates to the detection of the improved phenotypic seed quality characteristics by analysis of the polymorphism of an Sh2 gene in order to select plants with improved seed quality, and also to means for implementing this detection.

### PRIOR ART

Grain Composition

The grain accumulates sufficient energy stores to allow germination of the embryo. These stores are in the form of carbohydric, protein or oleic stores. In cereals, the accumulated stores are mainly in carbohydrate and protein forms. The carbohydrate stores consist in large part of polysaccharides such as starch. Starch is made up of two distinct polysaccharide fractions, amylose and amylopectin. Amylose constitutes the minor fraction of starch; it is a chain of α1-4-linked glucose monomers exhibiting less than 1% branching. Amylopectin represents the major fraction of starch. It consists of α1-4-linked glucose monomers and is approximately 5% branched with glucose monomers linked to the main chain by α1-6 linkages. Starch represents close to 85% by weight of the albumen of grains, the remaining energy store fraction consisting essentially of proteins.

Agronomic and Nutritive Importance of Cereals and of Starch

Starch is the major polysaccharide of energy storage in plants. It is of particular importance in cereals. Thus, rice starch, wheat starch or maize starch constitutes the main source of sugar intake of the human or animal diet. The study of grain filling, and the selection and creation of cereal varieties with modified starch contents constitute one of the favored areas of research concerning cereals.

ADP-Glucose Pyrophosphorylase Enzyme

ADP glucose pyrophosphorylase (AGPase) is a key enzyme of the polysaccharide biosynthesis pathway, both in bacteria and in plants. In plants, it makes it possible to dephosphorylate glucose-1-phosphate to ADP-glucose, the monomer constituting starch.

Plant AGPase has a complex structure in the form of a heterotetramer made up of two types of similar but distinct subunits. The two types of subunits, having a molecular weight close to 50 kDa in the endosperm, are encoded, for one, by the Sh2, for Shrunken 2, gene (Bhave et al., 1990) and, for the other, by the Bt2, for Brittle-2, gene (Bae et al., 1990).

Genetic Polymorphism

In the same species, the genome exhibits polymorphism. The main causes of these genetic variabilities are mutations which occur during the DNA replication preceding cell multiplication. This may be the insertion or the deletion of one or more nucleotides (included in the term “indel”), or else a substitution of the transition type (substitution of a purine base with another purine base or of a pyrimidine base with another pyrimidine base) or of the transversion type (substitution of a purine base with a pyrimidine base or vice versa). The polymorphic sites are thus classified into two categories depending on whether the substitution of a base (SNP for single nucleotide polymorphism) or else an indel (insertion-deletion) is involved.

While the polymorphic sites are found most commonly in the noncoding regions of the genome, some of the SNPs or of the indels are found in the coding regions of the genome or in regions involved in the regulation of transcription and/or of translation, such as the promoter regions, the 5′-leader regions, or even certain introns. This type of polymorphism can then have an impact on the expression of a gene, or the structure or the activity of a protein and, consequently, on the expression of a given phenotype by the plant.

The analysis of DNA sequence variability makes it possible to characterize an individual or a group of individuals by its genome. In fact, knowledge of the genetic variability of individuals makes it possible to generate markers for detecting the allelic form(s) present in other individuals. This may be PCR primers, allele-specific probes, or any other nucleotide sequence making it possible to detect a particular polymorphic site of the gene. When a marker specific to a locus is linked to a particular agronomic characteristic of the plant, knowledge of the allelic form of the marker makes it possible to predict the phenotype of an individual. It is also possible to use the markers defined by virtue of the sequence polymorphism to introgress or integrate, by transgenesis, a favorable allele into a plant.

In the Case of Maize, AGPase Enzyme and Sh2 Gene

In maize, several studies have revealed QTLs (for “Quantitative Trait Loci”) for grain filling characteristics, in the region of chromosome 3 which carries the Sh2 gene. They are in particular QTLs associated with starch and protein content (Goldman, 1993), and also with amylose content and with the amylose/amylopectin ratio (Séne et al., 2000). In addition, modifications of the 3′ upstream region of the gene, under the effect of transposons of the Ac/Ds type, can induce an increase in the amount of starch in the grain (Giroux et al. 1996). It therefore appears that the variability of the Sh2 gene may reflect the variation in grain filling characteristics. Shaw and Hannah (1992) have sequenced the Sh2 gene of the maize variety Black Mexican Sweet, which comprises approximately 7200 base pairs.

However, although the studies published in the state of the art on the Sh2 gene suggest a certain relationship between the activity of this gene and the qualities of the seed, they do not describe any precise technical means predictive of phenotypic seed quality characteristics, which can be used on a large scale and which make it possible to discriminate, with respect to one another, the various types or levels of phenotype characterizing seed quality, such as the number of grains per ear, the mass of the mature grain, the starch content of the grain, the amylose content of the grain or the protein content of the grain, or else a combination of the abovementioned phenotypic characteristics.

### SUMMARY OF THE INVENTION

The invention provides, for the first time, a set of means for selecting plant varieties for their improved phenotypic seed quality characteristics by analyzing polymorphisms newly identified in the Sh2 gene, which polymorphisms are statistically associated with a precise phenotypic seed quality characteristic, or with a combination of phenotypic seed quality characteristics.

It has in fact been shown, according to the invention, that a given allele of each of the new polymorphic sites of the Sh2 gene is statistically associated with the expression of one or more phenotypic characteristics defining seed quality.

A subject of the invention is the use of a nucleotide probe or primer in a process for selecting plants having improved phenotypic seed quality characteristics, characterized in that said nucleotide probe or said nucleotide primer allows the detection of a polymorphic base or of a polymorphic nucleotide sequence defining an allele of a polymorphic site of the Sh2 gene of sequence SEQ ID No. 1, said polymorphic base or said polymorphic nucleotide sequence being contained in a nucleic acid included in the Sh2 gene, chosen from the nucleic acids comprising a polymorphic nucleotide site associated with a characteristic or a combination of phenotypic characteristics linked to seed quality, in particular the number of seeds per ear, the mass of the mature seed, the protein content of the seed, the starch content of the seed, the amylose content of the seed or the protein/starch weight ratio in the seed.

It also relates to a process for determining the identity of the allele of a polymorphic site within a nucleic acid derived from an Sh2 gene for the purpose of selecting a plant having improved phenotypic seed quality characteristics, characterized in that it comprises a step consisting of characterizing the identity of the polymorphic base or of the polymorphic nucleotide sequence present at at least one nucleotide position of said nucleic acid corresponding to at least one of the nucleotides included in a newly identified polymorphic nucleotide site of the Sh2 gene.

According to this process, the determination of the identity of the allele of a polymorphic site or of a combination of polymorphic sites makes it possible to predict the seed quality phenotype of the analyzed plant, without requiring direct analysis of the phenotypic characteristics themselves.

The invention also relates to nucleotide probes and primers for determining the allelic form of a polymorphic site of the Sh2 gene, useful in particular as means for determining the identity of the polymorphic base or of the polymorphic nucleotide sequence at the polymorphic site associated with a phenotypic seed quality characteristic or with a combination of phenotypic seed quality characteristics.

A subject of the invention is also a nucleic acid derived from the Sh2 gene and comprising at least one polymorphic site as defined in the present description, and also recombinant vectors comprising such a nucleic acid.

The invention also relates to a host cell transformed with a nucleic acid or a recombinant vector described above, preferably a bacterial or plant host cell.

It also relates to a plant transformed with a nucleic acid or with a recombinant vector described above.

It also relates to antibodies directed specifically against an SH2 polypeptide encoded by a nucleic acid derived from the Sh2 gene comprising a polymorphic site as defined in the present description, and also to a pack or kit comprising one of these antibodies or else a combination of several of these antibodies.

### DESCRIPTION OF THE FIGURES

FIG. 1 represents the nucleotide sequence of the Sh2 gene described by Shaw and Hannah (1992), which is identical to the sequence SEQ ID No. 1 of the sequence listing.

The first column from the left represents the nucleotide numbering of the sequence SEQ ID No. 1 of the sequence listing of the present description. The first nucleotide is numbered 1.

The second column from the left represents the nucleotide numbering recommended by Shaw and Hannah (1992), which is based on the position of the nucleotide of the transcription initiation site, numbered +1.

The nucleotide at position 1 of the sequence SEQ ID No. 1 is the nucleotide at position −1020 according to the nomenclature of Shaw and Hannah (1992).

### DETAILED DESCRIPTION OF THE INVENTION

The applicant has identified, in the sequence of the Sh2 gene, a collection of polymorphisms for which it has shown that they are each associated with at least one phenotypic characteristic defining the seed quality of a plant. The identification of these polymorphisms has made it possible to develop processes for detecting these polymorphisms using the DNA of a plant individual, plant cell, plant at the plantlet stage, plant at an early stage of development or plant at the vegetative stage, making it possible to predict which are or which will be the phenotypic characteristics linked to the quality of the seed or of the future seed.

The association between the presence of a defined allele of a polymorphic site of the Sh2 gene and at least one grain filling characteristic makes it possible to define the relationship between a given allele or a combination of given alleles (haplotype) and a phenotypic characteristic or a combination of phenotypic characteristics defining the seed quality. The inventors have shown that the polymorphism of the Sh2 gene is statistically associated with seed quality characteristics such as:
the number of seeds per ear
the mass of the mature seed
the protein content of the seed
the starch content of the seed
the amylose content of the seed
the protein content/starch content ratio in the seed.

The identification of specific alleles of polymorphic sites associated with seed quality characteristics, in the sequence of the Sh2 gene, has made it possible to define oligonucleotide sequences specific for a given allele of one or more polymorphic sites of the Sh2 gene and to use these sequences as probes or primers for facilitating the marker-assisted selection of plants, for generating favorable allelic sequences by site-directed mutagenesis, or for cloning sequences which introduce a favorable agronomic characteristic, linked to the seed quality, into the plant transformed with these sequences.

It has also been shown, according to the invention, that certain polymorphisms of the Sh2 gene result in modifications in the amino acid sequence. The favorable alleles detected in these polymorphisms can be used to modify the structure or the activity of the AGPase enzyme.

The Sh2 gene used as reference nucleotide sequence, based on which the various newly identified polymorphic sites according to the invention are defined, is the nucleotide sequence referenced in the GenBank database under the accession number M81603, and which is reproduced as the sequence SEQ ID No. 1 in the sequence listing.

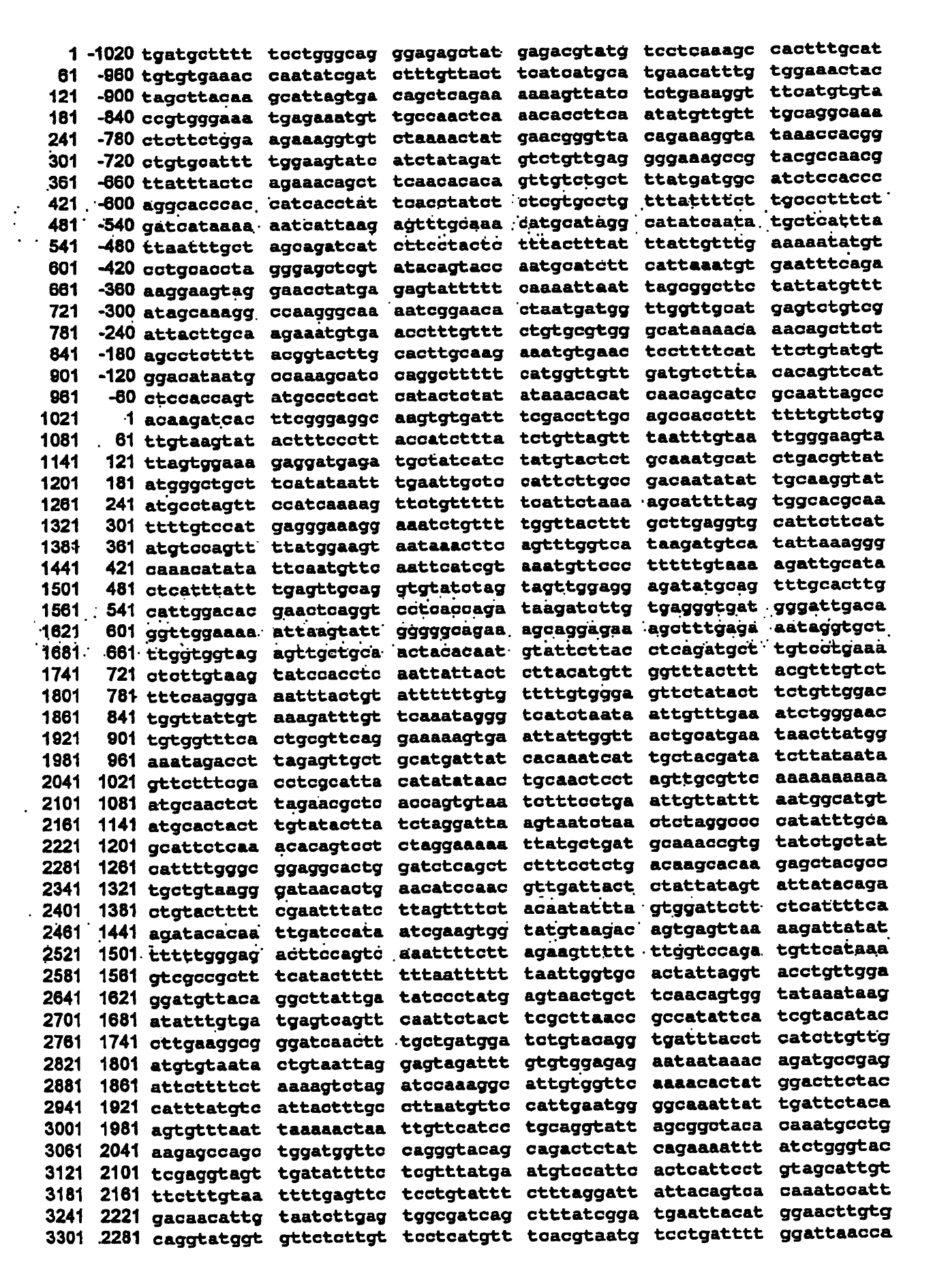

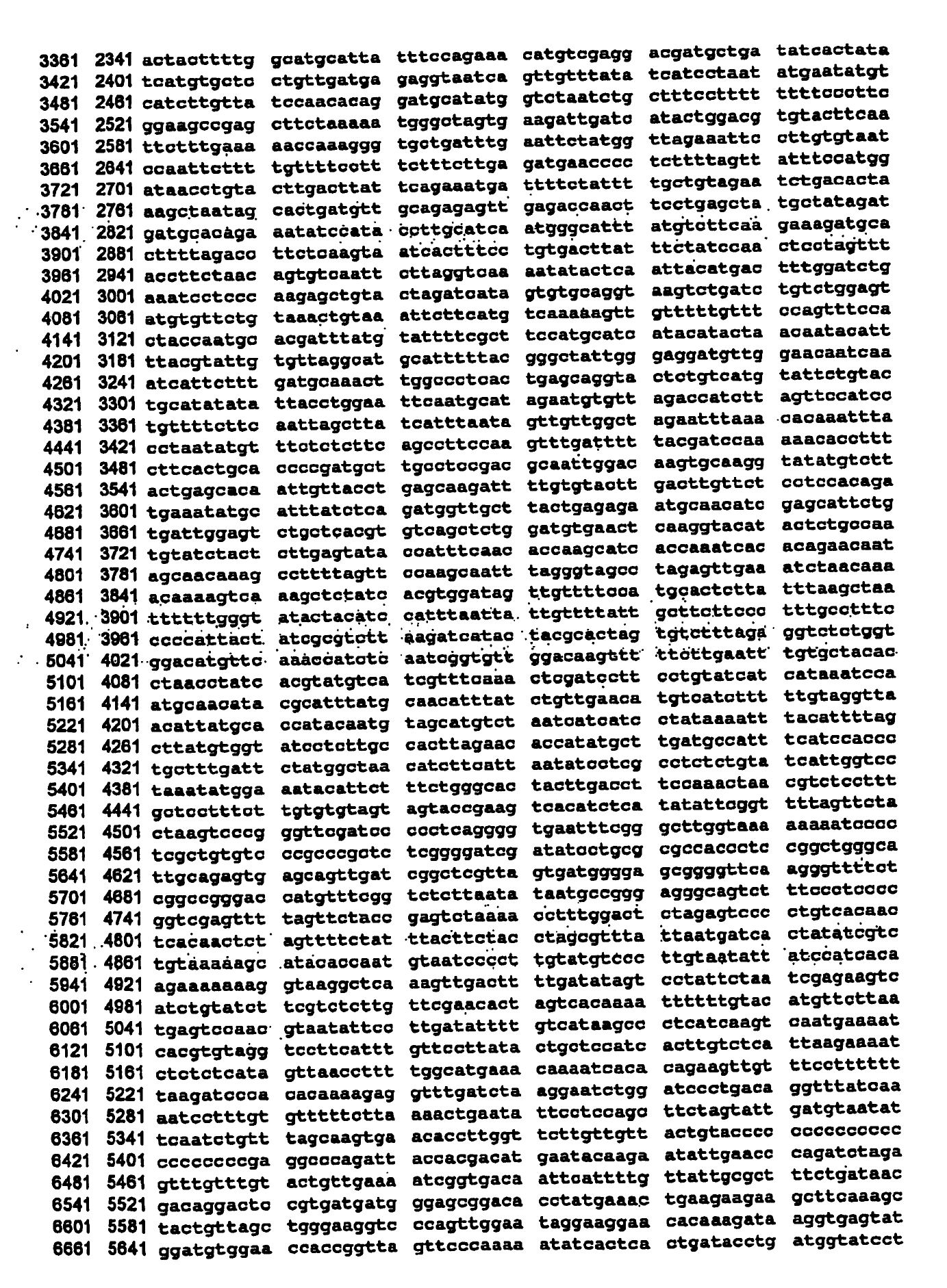

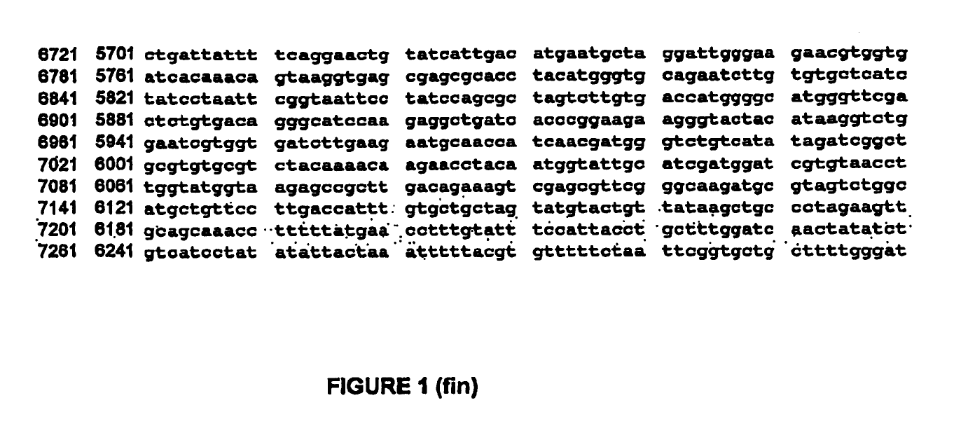

The Sh2 gene of sequence SEQ ID No. 1 is 7320 nucleotides in length. It has 16 exons, respectively:
Exon No. 1: from the nucleotide at position 1021 up to the nucleotide at position 1082 of the sequence SEQ ID No. 1, which corresponds to the sequence ranging from the nucleotide at position 1 up to the nucleotide at position 62 of the Sh2 gene according to the nomenclature of Shaw and Hannah (1992), as illustrated in FIG. 1;
Exon No. 2: from the nucleotide at position 1521 up to the nucleotide at position 1745 of the sequence SEQ ID No. 1, which corresponds to the sequence ranging from the nucleotide at position 501 up to the nucleotide at position 725 of the Sh2 gene according to the nomenclature of Shaw and Hannah (1992);
Exon No. 3: from the nucleotide at position 2222 up to the nucleotide at position 2344 of the sequence SEQ ID No. 1, which corresponds to the sequence ranging from the nucleotide at position 1202 up to the nucleotide at position 1324 of the Sh2 gene according to the nomenclature of Shaw and Hannah (1992);
Exon No. 4: from the nucleotide at position 2629 up to the nucleotide at position 2799 of the sequence SEQ ID No. 1, which corresponds to the sequence ranging from the nucleotide at position 1609 up to the nucleotide at position 1779 of the Sh2 gene according to the nomenclature of Shaw and Hannah (1992);
Exon No. 5: from the nucleotide at position 3036 up to the nucleotide at position 3125 of the sequence SEQ ID No. 1, which corresponds to the sequence ranging from the nucleotide at position 2016 up to the nucleotide at position 2105 of the Sh2 gene according to the nomenclature of Shaw and Hannah (1992);
Exon No. 6: from the nucleotide at position 3217 up to the nucleotide at position 3303 of the sequence SEQ ID No. 1, which corresponds to the sequence ranging from the nucleotide at position 2197 up to the nucleotide at position 2283 of the Sh2 gene according to the nomenclature of Shaw and Hannah (1992);
Exon No. 7: from the nucleotide at position 3388 up to the nucleotide at position 3443 of the sequence SEQ ID No. 1, which corresponds to the sequence ranging from the nucleotide at position 2368 up to the nucleotide at position 2423 of the Sh2 gene according to the nomenclature of Shaw and Hannah (1992);
Exon No. 8: from the nucleotide at position 3546 up to the nucleotide at position 3639 of the sequence SEQ ID No. 1, which corresponds to the sequence ranging from the nucleotide at position 2526 up to the nucleotide at position 2619 of the Sh2 gene according to the nomenclature of Shaw and Hannah (1992);
Exon No. 9: from the nucleotide at position 3805 up to the nucleotide at position 3917 of the sequence SEQ ID No. 1, which corresponds to the sequence ranging from the nucleotide at position 2785 up to the nucleotide at position 2897 of the Sh2 gene according to the nomenclature of Shaw and Hannah (1992);
Exon No. 10: from the nucleotide at position 3986 up to the nucleotide at position 4058 of the sequence SEQ ID No. 1, which corresponds to the sequence ranging from the nucleotide at position 2966 up to the nucleotide at position 3038 of the Sh2 gene according to the nomenclature of Shaw and Hannah (1992);
Exon No. 11: from the nucleotide at position 4217 up to the nucleotide at position 4297 of the sequence SEQ ID No. 1, which corresponds to the sequence ranging from the nucleotide at position 3197 up to the nucleotide at position 3277 of the Sh2 gene according to the nomenclature of Shaw and Hannah (1992);
Exon No. 12: from the nucleotide at position 4463 up to the nucleotide at position 4549 of the sequence SEQ ID No. 1, which corresponds to the sequence ranging from the nucleotide at position 3443 up to the nucleotide at position 3529 of the Sh2 gene according to the nomenclature of Shaw and Hannah (1992);
Exon No. 13: from the nucleotide at position 4620 up to the nucleotide at position 4724 of the sequence SEQ ID No. 1, which corresponds to the sequence ranging from the nucleotide at position 3600 up to the nucleotide at position 3704 of the Sh2 gene according to the nomenclature of Shaw and Hannah (1992);
Exon No. 14: from the nucleotide at position 6546 up to the nucleotide at position 6652 of the sequence SEQ ID No. 1, which corresponds to the sequence ranging from the nucleotide at position 5526 up to the nucleotide at position 5632 of the Sh2 gene according to the nomenclature of Shaw and Hannah (1992);
Exon No. 15: from the nucleotide at position 6735 up to the nucleotide at position 6795 of the sequence SEQ ID No. 1, which corresponds to the sequence ranging from the nucleotide at position 5715 up to the nucleotide at position 5775 of the Sh2 gene according to the nomenclature of Shaw and Hannah (1992);
Exon No. 16: from the nucleotide at position 6912 up to the nucleotide at position 7287 of the sequence SEQ ID No. 1, which corresponds to the sequence ranging from the nucleotide at position 5892 up to the nucleotide at position 6267 of the Sh2 gene according to the nomenclature of Shaw and Hannah (1992).

In the present description, the numbering used to define the position of the polymorphic nucleotide or of the polymorphic nucleotide sequence of a polymorphic site of the Sh2 gene is exclusively that described by Shaw and Hannah (1992) and which is recalled in the first column from the left of the sequence of the Sh2 gene represented in FIG. 1.

Polymorphic Sites of the Sh2 Gene According to the Invention

The polymorphism of the Sh2 gene was determined from the DNA originating from 33 inbred maize lines, whose phenotypic characteristics linked to grain quality were simultaneously analyzed.

Polymorphism analysis has made it possible to characterize 72 polymorphic sites in the sequence of the Sh2 gene.

Among these 72 polymorphic sites, 19 of them exhibited a nucleotide difference for just one of the lines studied. These 19 polymorphic sites did not exhibit an informative statistical distribution which could be used in a study of association between the presence of a given allele of these polymorphic sites and the observation of a given phenotypic seed quality characteristic.

Among the 53 informative polymorphic sites, some of them were considered to be redundant in that the same alleles of two or more of these sites were systematically found jointly in the DNA of several lines. Two redundant polymorphic sites provide the same statistical information concerning the association of one of their alleles with a given phenotypic seed quality characteristic. They enable an identical discrimination between two lines or two groups of lines.

According to the invention, 43 polymorphic sites of the Sh2 gene were selected as constituting polymorphisms or groups of polymorphisms of interest, a given and characterized allele of which is statistically associated with a phenotypic seed quality characteristic and sometimes with several phenotypic seed quality characteristics.

The term “polymorphic site” of the Sh2 gene is intended to mean a region of the sequence of the Sh2 gene which, in the genome of certain plant lines, more specifically in certain maize lines, differs by one or more consecutive nucleotides compared to the corresponding region of the Sh2 gene defined by the sequence SEQ ID No. 1. A polymorphic site may consist of a variation of a single nucleotide which can have two meanings, for example a base A or a base T, such a polymorphic site then being referred to as an SNP (for “Single Nucleotide Polymorphism”). A polymorphic site may also consist of the addition or the deletion of one or more consecutive nucleotides compared to the reference sequence SEQ ID No. 1. This second type of polymorphic site is referred to as “indel” (for “insertion-deletion”).

The polymorphic sites of the Sh2 gene characterized in the present invention are defined below. They are characterized by their nucleotide difference compared to the reference Sh2 gene sequence SEQ ID No. 1, the nucleotide positions of which are defined according to the nomenclature of Shaw and Hannah (1992). Such as they are defined below, the polymorphic sites of the Sh2 gene are characterized by their allele whose presence, in the genome of a plant, is associated with the expression of a modified phenotypic seed quality characteristic.
(a) a nucleic acid in which the nucleotide corresponding to the nucleotide at position −921 of the Sh2 gene is a G;
(b) a nucleic acid in which the nucleotides corresponding to the nucleotides at positions −830 to −824, of sequence 5′-TGAGAAA-3′, of the Sh2 gene are absent;
(c) a nucleic acid in which the nucleotides corresponding to the nucleotides at positions −580 to −573, of sequence 5′-TCACCTAT-3′, of the Sh2 gene are absent;
(d) a nucleic acid in which the nucleotide corresponding to the nucleotide at position −438 of the Sh2 gene is a G;
(e) a nucleic acid in which the nucleotide corresponding to the nucleotide at position −362 of the Sh2 gene is an A;
(f) a nucleic acid in which the nucleotide corresponding to the nucleotide at position −347 of the Sh2 gene is a T;
(g) a nucleic acid in which the nucleotide corresponding to the nucleotide at position −296 of the Sh2 gene is a T;
(h) a nucleic acid in which the nucleotide corresponding to the nucleotide at position −277 of the Sh2 gene is a T;
(i) a nucleic acid in which the nucleotide corresponding to the nucleotide at position −266 of the Sh2 gene is a C;
(j) a nucleic acid in which the nucleotide corresponding to the nucleotide at position −168 of the Sh2 gene is an A;
(k) a nucleic acid in which the nucleotide corresponding to the nucleotide at position −15 of the Sh2 gene is an A;
(l) a nucleic acid in which the nucleotide corresponding to the nucleotide at position +35 of the Sh2 gene is a T;
(m) a nucleic acid in which an additional T is found after the nucleotide at position +304 of the Sh2 gene;
(n) a nucleic acid in which the nucleotide corresponding to the nucleotide at position +515 of the Sh2 gene is a C;
(o) a nucleic acid in which the nucleotide corresponding to the nucleotide at position +587 of the Sh2 gene is a C;
(p) a nucleic acid in which the nucleotide corresponding to the nucleotide at position +678 of the Sh2 gene is an A;
(q) a nucleic acid in which the nucleotide corresponding to the nucleotide at position +960 of the Sh2 gene is an A;
(r) a nucleic acid in which the nucleotide corresponding to the nucleotide at position +1059 of the Sh2 gene is a G;
(s) a nucleic acid in which the nucleotide corresponding to the nucleotide at position +1068 of the Sh2 gene is a G;
(t) a nucleic acid in which the nucleotide A corresponding to the nucleotide at position +1081 of the Sh2 gene is absent;
(u) a nucleic acid in which the nucleotide corresponding to the nucleotide at position +1473 of the Sh2 gene is a C;
(v) a nucleic acid in which an additional T is present after the nucleotide at position +1505 of the Sh2 gene;
(w) a nucleic acid in which an additional T is present after the nucleotide at position +1542 of the Sh2 gene;
(x) a nucleic acid in which the nucleotide corresponding to the nucleotide at position +1867 of the Sh2 gene is a C;
(y) a nucleic acid in which the nucleotide T corresponding to the nucleotide at position +2514 of the Sh2 gene is absent;
(z) a nucleic acid in which an additional T is present after the nucleotide at position 2771 of the Sh2 gene;
(ab) a nucleic acid in which the nucleotide corresponding to the nucleotide at position +2939 of the Sh2 gene is a G;
(ac) a nucleic acid in which the nucleotide corresponding to the nucleotide at position +2983 of the Sh2 gene is a C; and
(ad) a nucleic acid comprising the insertion of the sequence 5′-GTTTTTATTTA-3′ after the nucleotide corresponding to the nucleotide at position +3123 of the Sh2 gene.

For the polymorphic site +587, the presence of a base C constitutes a conservative base substitution which does not result in any change in the amino acid sequence of the corresponding SH2 polypeptide, compared to the SH2 polypeptide of sequence SEQ ID No. 52 encoded by the sequence SEQ ID No. 1.

For the polymorphic site +678, the presence of a base A results in the replacement, in the sequence of the SH2 polypeptide of sequence SEQ ID No. 52, of the alanine amino acid, encoded by the sequence SEQ ID No. 1 at this position, with a threonine amino acid.

For the polymorphic site +2983, the presence of a base C results in the replacement, in the sequence of the SH2 polypeptide of sequence SEQ ID No. 52, of the leucine amino acid, encoded by the sequence SEQ ID No. 1 at this position, with a serine amino acid.

As previously set out, the characterization according to the invention of informative polymorphic sites within the sequence of the Sh2 gene has made it possible to construct various means for detecting a given allele of each of the polymorphic sites, useful in particular in processes for selecting plant lines, specifically maize lines, having desired phenotypic seed quality characteristics.

Such plant line selection processes can be carried out at early stages of plant development, before seed formation, at a time in the development of the plant for which it is not possible to determine the phenotypic characteristics of the seed. The processes using an analysis of the polymorphic sites of the Sh2 gene of the invention therefore have a predictive value of great technical and economic interest.

Uses and Processes Using the Characteristics of the Polymorphic Sites According to the Invention.

A subject of the invention is the use of a nucleotide probe or of a nucleotide primer in a process for selecting plants having improved phenotypic seed quality characteristics, characterized in that said nucleotide probe or said nucleotide primer makes it possible to detect a polymorphic base or a polymorphic nucleotide sequence defining an allele of a polymorphic site of the Sh2 gene of sequence SEQ ID No. 1, said polymorphic base or said polymorphic nucleotide sequence being contained in a nucleic acid included in an Sh2 gene, chosen from the following nucleic acids:
(a) a nucleic acid in which the nucleotide corresponding to the nucleotide at position −921 of the Sh2 gene is a G;
(b) a nucleic acid in which the nucleotides corresponding to the nucleotides at positions −830 to −824, of sequence 5′-TGAGAAA-3′, of the Sh2 gene are absent;
(c) a nucleic acid in which the nucleotides corresponding to the nucleotides at positions −580 to −573, of sequence 5′-TCACCTAT-3′, of the Sh2 gene are absent;
(d) a nucleic acid in which the nucleotide corresponding to the nucleotide at position −438 of the Sh2 gene is a G;
(e) a nucleic acid in which the nucleotide corresponding to the nucleotide at position −362 of the Sh2 gene is an A;
(f) a nucleic acid in which the nucleotide corresponding to the nucleotide at position −347 of the Sh2 gene is a T;
(g) a nucleic acid in which the nucleotide corresponding to the nucleotide at position −296 of the Sh2 gene is a T;
(h) a nucleic acid in which the nucleotide corresponding to the nucleotide at position −277 of the Sh2 gene is a T;
(i) a nucleic acid in which the nucleotide corresponding to the nucleotide at position −266 of the Sh2 gene is a C;
(j) a nucleic acid in which the nucleotide corresponding to the nucleotide at position −168 of the Sh2 gene is an A;
(k) a nucleic acid in which the nucleotide corresponding to the nucleotide at position −15 of the Sh2 gene is an A;
(l) a nucleic acid in which the nucleotide corresponding to the nucleotide at position +35 of the Sh2 gene is a T;
(m) a nucleic acid in which an additional T is found after the nucleotide at position +304 of the Sh2 gene;
(n) a nucleic acid in which the nucleotide corresponding to the nucleotide at position +515 of the Sh2 gene is a C;
(o) a nucleic acid in which the nucleotide corresponding to the nucleotide at position +587 of the Sh2 gene is a C;
(p) a nucleic acid in which the nucleotide corresponding to the nucleotide at position +678 of the Sh2 gene is an A;
(q) a nucleic acid in which the nucleotide corresponding to the nucleotide at position +960 of the Sh2 gene is an A;
(r) a nucleic acid in which the nucleotide corresponding to the nucleotide at position +1059 of the Sh2 gene is a G;
(s) a nucleic acid in which the nucleotide corresponding to the nucleotide at position +1068 of the Sh2 gene is a G;
(t) a nucleic acid in which the nucleotide A corresponding to the nucleotide at position +1081 of the Sh2 gene is absent;
(u) a nucleic acid in which the nucleotide corresponding to the nucleotide at position +1473 of the Sh2 gene is a C;
(v) a nucleic acid in which an additional T is present after the nucleotide at position +1505 of the Sh2 gene;
(w) a nucleic acid in which an additional T is present after the nucleotide at position +1542 of the Sh2 gene;
(x) a nucleic acid in which the nucleotide corresponding to the nucleotide at position +1867 of the Sh2 gene is a C;
(y) a nucleic acid in which the nucleotide T corresponding to the nucleotide at position +2514 of the Sh2 gene is absent;
(z) a nucleic acid in which an additional T is present after the nucleotide at position 2771 of the Sh2 gene;
(ab) a nucleic acid in which the nucleotide corresponding to the nucleotide at position +2939 of the Sh2 gene is a G;
(ac) a nucleic acid in which the nucleotide corresponding to the nucleotide at position +2983 of the Sh2 gene is a C; and
(ad) a nucleic acid comprising the insertion of the sequence 5′-GTTTTTATTTA-3′ after the nucleotide corresponding to the nucleotide at position +3123 of the Sh2 gene.

The expression “grain quality” encompasses the characteristics which influence the quality and the quantity of the seed, such as, for example: the number of seeds per ear, the mass of the mature seeds, the protein content, starch content and amylose content, and the protein content/starch content weight ratio in the seed, and also all the complex characteristics including one or more of these characteristics.

One of the methods which can be used to determine the phenotype of an individual at this or these sites may consist in:
i) obtaining a DNA sample from an individual,
ii) identifying the allele at one or more of the abovementioned positions (with reference to the positions described according to the Genbank sequence No. M81603) in the Sh2 gene,
iii) predicting the grain quality of the individual with reference to the association of the polymorphism of the Sh2 gene as described above and the grain quality characteristic.

The DNA sample is obtained according to the conventional methods of DNA extraction used in plants, in particular from young maize leaves (Dellaporta et al. 1983).

The DNA sample would have to contain at least the sequence of the Sh2 gene, i.e. a region of this sequence which can be amplified by any technique known from the prior art, such as, for example, PCR.

The grain quality of the individual studied can be determined with reference to the presence of an allele at at least one, several or all of the positions described, in combination with other polymorphisms in the Sh2 gene which are known or which will subsequently be characterized.

There are a large number of known analytical procedures in the state of the art which can be used by those skilled in the art for detecting the allelic form of one or more polymorphic sites of the Sh2 gene of the invention. In general, the detection of alleles requires a polymorphism discriminating technique. In general, common methods involve amplification of the target sequence and then identification of the alleles by short-probe hybridizations, by endonuclease restriction, by discrimination with polymerases or ligases, observing the nucleotide incorporated by the polymerase as a function of the matrix sequence, or by detection of mismatching on the double-stranded DNA.

Among these techniques for detecting alleles, the following techniques may be mentioned: DNA sequencing, sequencing by hybridization, SSCP (Single strand conformation polymorphism analysis), DGGE (Denaturing gradient gel electrophoresis), TGGE (Temperature gradient gel electrophoresis), heteroduplex analysis, CMC (Chemical mismatch cleavage), dot blots, reverse dot blots, oligonucleotide array Taqman™ (U.S. Pat. No. 5,210,015 and U.S. Pat. No. 5,487,972, Hoffmann-La Roche), ARMS™ (Amplification refractory mutation system), ALEX™ (Amplification refractory mutation system linear extension, EP 332435B1, Zeneca Ltd), COPS (Gibbs et al. 1989), mini-sequencing, APEX (Arrayed primer extension), RFLP (Restriction fragment length polymorphism), cleaved amplified polymorphic sequences (CAPS), OLA (Oligonucleotide ligation assay), pyrosequencing (Nyrèn et al. 1997).

These techniques can be used in combination with signal-generating systems such as, for example, the following techniques: FRET (Fluorescence resonance energy transfer), fluorescence quenching, fluorescence polarization (UK 2228998, Zeneca Ltd), chemiluminescence, electrochemiluminescence, radioactivity, colorimetry, hybridization protection assay, mass spectrometry. Other amplification techniques: such as SSR (Self sustained replication), LCR (Ligase chain reaction), SDA (Strand displacement amplification) or leb-DNA (branched DNA). Most of these techniques for detecting allelic variations are recapitulated in standard works such as “Laboratory Protocols for mutation detection”, Ed. by U. Landegren, Oxford University Press, 1996 and “PCR”, 2^{nd} Edition by Newton and Graham, BIOS Scientific Publishers Ltd, 1997). Other protocols for sequencing and cloning and other aspects of molecular biology are listed in “Genes VII” (Lewin, 1999).

These techniques can use oligonucleotides synthesized from the following sequences (the allelic form of the SNP or indel detected in the invention is indicated between brackets, and the difference between the allelic form of the reference variety BMS and the allele described is indicated in bold):

In the use of a nucleotide probe or of a nucleotide primer, as defined above, said nucleotide probe or said nucleotide primer can be characterized in that it makes it possible to discriminate between the presence of a first nucleic acid (1) and of a second nucleic acid (2), said nucleic acids (1) and (2) being chosen from the following:

Polymorphic Sites
(a) Site −921: the nucleic acid (1) of sequence SEQ ID No. 2 in which the nucleotide at position 41 is a base G and the nucleic acid (2) of sequence SEQ ID No. 2 in which the nucleotide at position 41 is a base A;
(b) Site −438: the nucleic acid (1) of sequence SEQ ID No. 3 in which the nucleotide at position 41 is a base G and the nucleic acid (2) of sequence SEQ ID No. 3 in which the nucleotide at position 41 is a base A;
(c) Site −362: the nucleic acid (1) of sequence SEQ ID No. 4 in which the nucleotide at position 41 is a base A and the nucleic acid (2) of sequence SEQ ID No. 4 in which the nucleotide at position 41 is a base G;
(d) Site −347: the nucleic acid (1) of sequence SEQ ID No. 5 in which the nucleotide at position 41 is a base T and the nucleic acid (2) of sequence SEQ ID No. 5 in which the nucleotide at position 41 is a base C;
(e) Site −296: the nucleic acid (1) of sequence SEQ ID No. 6 in which the nucleotide at position 41 is a base T and the nucleic acid (2) of sequence SEQ ID No. 6 in which the nucleotide at position 41 is a base C;
(f) Site −277: the nucleic acid (1) of sequence SEQ ID No. 7 in which the nucleotide at position 41 is a base T and the nucleic acid (2) of sequence SEQ ID No. 7 in which the nucleotide at position 41 is a base C;
(g) Site −266: the nucleic acid (1) of sequence SEQ ID No. 8 in which the nucleotide at position 41 is a base C and the nucleic acid (2) of sequence SEQ ID No. 8 in which the nucleotide at position 41 is a base T;
(h) Site −168: the nucleic acid (1) of sequence SEQ ID No. 9 in which the nucleotide at position 41 is a base A and the nucleic acid (2) of sequence SEQ ID No. 9 in which the nucleotide at position 41 is a base G;
(i) Site −15: the nucleic acid (1) of sequence SEQ ID No. 10 in which the nucleotide at position 41 is a base A and the nucleic acid (2) of sequence SEQ ID No. 10 in which the nucleotide at position 41 is a base G;
(j) Site +35: the nucleic acid (1) of sequence SEQ ID No. 11 in which the nucleotide at position 41 is a base T and the nucleic acid (2) of sequence SEQ ID No. 11 in which the nucleotide at position 41 is a base C;
(k) Site +515: the nucleic acid (1) of sequence SEQ ID No. 12 in which the nucleotide at position 41 is a base C and the nucleic acid (2) of sequence SEQ ID No. 12 in which the nucleotide at position 41 is a base T;
(l) Site +587: the nucleic acid (1) of sequence SEQ ID No. 13 in which the nucleotide at position 41 is a base C and the nucleic acid (2) of sequence SEQ ID No. 13 in which the nucleotide at position 41 is a base T;
(m) Site +678: the nucleic acid (1) of sequence SEQ ID No. 14 in which the nucleotide at position 41 is a base A and the nucleic acid (2) of sequence SEQ ID No. 14 in which the nucleotide at position 41 is a base G;
(n) Site +960: the nucleic acid (1) of sequence SEQ ID No. 15 in which the nucleotide at position 41 is a base A and the nucleic acid (2) of sequence SEQ ID No. 15 in which the nucleotide at position 41 is a base G;
(o) Site +1059: the nucleic acid (1) of sequence SEQ ID No. 16 in which the nucleotide at position 41 is a base G and the nucleic acid (2) of sequence SEQ ID No. 16 in which the nucleotide at position 41 is a base C;
(p) Site +1068: the nucleic acid (1) of sequence SEQ ID No. 17 in which the nucleotide at position 41 is a base G and the nucleic acid (2) of sequence SEQ ID No. 17 in which the nucleotide at position 41 is a base T;
(q) Site +1473: the nucleic acid (1) of sequence SEQ ID No. 18 in which the nucleotide at position 41 is a base C and the nucleic acid (2) of sequence SEQ ID No. 18 in which the nucleotide at position 41 is a base T;
(r) Site +1867: the nucleic acid (1) of sequence SEQ ID No. 19 in which the nucleotide at position 41 is a base C and the nucleic acid (2) of sequence SEQ ID No. 19 in which the nucleotide at position 41 is a base T;
(s) Site +2939: the nucleic acid (1) of sequence SEQ ID No. 20 in which the nucleotide at position 41 is a base G and the nucleic acid (2) of sequence SEQ ID No. 20 in which the nucleotide at position 41 is a base T;
(t) Site +2983: the nucleic acid (1) of sequence SEQ ID No. 21 in which the nucleotide at position 41 is a base C and the nucleic acid (2) of sequence SEQ ID No. 21 in which the nucleotide at position 41 is a base T;
(u) Site −830 to −824: the nucleic acid (1) of sequence SEQ ID No. 23 and the nucleic acid (2) of sequence SEQ ID No. 22;
(v) Site −580 to −573: the nucleic acid (1) of sequence SEQ ID No. 25 and the nucleic acid (2) of sequence SEQ ID No. 24;
(w) Site +304: the nucleic acid (1) of sequence SEQ ID No. 27 and the nucleic acid (2) of sequence SEQ ID No. 26;
(x) Site +1081: the nucleic acid (1) of sequence SEQ ID No. 29 and the nucleic acid (2) of sequence SEQ ID No. 28;
(y) Site +1505: the nucleic acid (1) of sequence SEQ ID No. 31 and the nucleic acid (2) of sequence SEQ ID No. 30;
(z) Site +1542: the nucleic acid (1) of sequence SEQ ID No. 33 and the nucleic acid (2) of sequence SEQ ID No. 32;
(aa) Site +2514: the nucleic acid (1) of sequence SEQ ID No. 35 and the nucleic acid (2) of sequence SEQ ID No. 34;
(ab) Site +2771: the nucleic acid (1) of sequence SEQ ID No. 37 and the nucleic acid (2) of sequence SEQ ID No. 36; et
(ac) Site +3123: the nucleic acid (1) of sequence SEQ ID No. 39 and the nucleic acid (2) of sequence SEQ ID No. 38.

According to another preferred embodiment, said nucleic acid is a nucleic acid which hybridizes specifically with a nucleic acid of sequence complementary to any one of the nucleic acids (1) or (2) defined in (a) to (ac) above.

The use above can also be characterized in that:
a) the nucleotide probe hybridizes specifically with a nucleic acid of a first allelic form of the polymorphic base or of the polymorphic nucleotide sequence defining a first allele of a polymorphic site of the Sh2 gene and does not hybridize with a nucleic acid of a second allelic form of the polymorphic base or of the polymorphic nucleotide sequence defining a second allele of a polymorphic site of the Sh2 gene; or
b) the nucleotide primer hybridizes specifically with a nucleotide sequence contained in an Sh2 gene, said nucleotide sequence being located upstream of an allelic form of a polymorphic base or of a polymorphic nucleotide sequence the presence or absence of which defines an allele of a polymorphic site of the Sh2 gene.

The polymorphism of the Sh2 gene, once identified by one of the methods described above, can be used, according to the invention, for carrying out a predictive selection of plants with a better grain quality, including the selection of plants at the plantlet stage and/or at the early stage and/or at the vegetative stage.

The nucleotide sequence of the maize Sh2 gene SEQ ID No. 1 exhibits strong nucleotide identity with the nucleotide sequences of many cereals, in particular of many grass varieties, which identity is virtually complete in the open reading frame (ORF).

In particular, the sequence of the maize Sh2 gene exhibits a very great nucleotide identity with the sorghum Sh2 gene, the greatest identity between the two sequences being found in the open reading frame (ORF). Thus, the SH2 polypeptide encoded by the sorghum Sh2 gene has a single amino acid difference with respect to the SH2 polypeptide encoded by the maize Sh2 gene SEQ ID No. 1.

Without wishing to be bound by any theory, the inventors think that the polymorphic sites found in the maize Sh2 gene are also found in the Sh2 gene of the genome of other cereals, in particular grasses, and even more specifically in the sorghum Sh2 gene. The polymorphic sites located in the open reading frame of the maize Sh2 gene SEQ ID No. 1 are those which have statistically the most probability of also being found in the Sh2 genes of other cereals, in particular of other grasses, such as sorghum.

The predictive selection therefore applies in particular to cereals such as, for example, maize and sorghum. The use of the allelic polymorphism described in the invention applies particularly to the selection of maize varieties. When a population is one segregating over several loci affecting several grain quality characteristics, marker-assisted selection (MAS) is more efficient than simple phenotypic evaluation since it in particular allows selection cycles which are much faster than the conventional techniques of direct characterization of the phenotype. Another advantage of MAS compared to phenotypic evaluation is that the analyses are completely independent of the vagaries of the weather.

The use above is also characterized in that the improved phenotypic seed quality characteristics are chosen from the number of seeds per ear, the seed mass, the protein content of the seeds, the starch content of the seeds, the amylose content of the seeds and the protein/starch weight ratio in the seeds, or a combination of these phenotypic characteristics.

A subject of the invention is also a process for determining the identity of the allele of a polymorphic site within a nucleic acid derived from an Sh2 gene for the purpose of selecting a plant having improved phenotypic seed quality characteristics, characterized in that it comprises a step consisting of characterizing the identity of the polymorphic base or of the polymorphic nucleotide sequence present at at least one nucleotide position of said nucleic acid corresponding to at least one of the nucleotides at position −921, −830 to −824, −580 to −573, −438, −362, −347, −296, −277, −266, −168, −15, +35, +304, +515, +587, +678, +960, +1059, +1068, +1081, +1473, +1505, +1542, +1867, +2514, +2771, +2939, +2983 and +3123 of the Sh2 gene of sequence SEQ ID No. 1.

According to a first aspect, the process above is characterized in that the characterization of the polymorphic site is carried out by sequencing said nucleic acid.

According to a second aspect of the process above, the characterization of the identity of the polymorphic site is carried out by hybridization of a nucleotide probe which hybridizes specifically with the sequence of a given allele of the Sh2 gene. According to this aspect, the characterization of the identity of the polymorphic site is carried out by hybridization of a nucleotide probe which hybridizes specifically with a polymorphic base or a polymorphic nucleotide sequence defining an allele of a given polymorphic site of the Sh2 gene.

According to a third aspect of the process above, the characterization of the polymorphic site is carried out by extension of a nucleotide primer which hybridizes specifically with a nucleotide sequence located upstream of an allelic form of a polymorphic base or of a polymorphic nucleotide sequence defining an allele of a given polymorphic site of an Sh2 gene.

According to a particular characteristic of this third aspect, the characterization of the identity of the polymorphic site is carried out by hybridization of a nucleotide primer which hybridizes specifically with the sequence located upstream, on the 5′ side of the polymorphic base or of the polymorphic nucleotide sequence defining a given allele of a polymorphic site of the Sh2 gene, and then extension of the primer. The characterization of the polymorphic allele can be carried out by sequencing of the primer extension product. In a particular embodiment, the nucleotide located at the 3′ end of the primer hybridizes with the nucleotide located immediately upstream on the 5′ side of the polymorphic base or of the polymorphic nucleotide sequence. In this particular embodiment, the identity of the allele of the polymorphic site considered can be determined directly by carrying out the primer extension step in the presence of fluorescent dideoxynucleotides which block the extension reaction. The identity of the dideoxynucleotide added to the sequence of the primer, and therefore the identity of the allele of the polymorphic site, is determined directly by fluorescence analysis. This is the microsequencing technique, well known in the state of the art. The primer hybridizes with the DNA strand comprising the sequence encoding the SH2 polypeptide (+ strand) or with the complementary DNA strand, which carries a base complementary to the corresponding base carried by the “+” strand at the polymorphic site.

According to a fourth aspect, the process is characterized in that, in order to select a plant having a modified number of seeds, the identity of the base or of a sequence of bases present at at least one nucleotide position of said nucleic acid corresponding to at least one of the nucleotides at position −168, +1473, +1542 and +2983 of the Sh2 gene of sequence SEQ ID No. 1 is determined.

According to a fifth aspect, the process is characterized in that, in order to select a plant with a modified seed mass, the identity of the base or of a sequence of bases present at at least one nucleotide position of said nucleic acid corresponding to at least one of the nucleotides at position −168, +1473, +1542 and +2983 of the Sh2 gene of sequence SEQ ID No. 1 is determined.

According to a sixth aspect, this process is characterized in that, in order to select a plant having a modified protein content in the seed, the identity of the base or of a sequence of bases present at at least one nucleotide position of said nucleic acid corresponding to at least one of the nucleotides at position −168, +1473, +1542, +2983, −830 to −824, −362, −347, −296, −15, +515, +587, +1068, +1505 and +2939 of the Sh2 gene of sequence SEQ ID No. 1 is determined.

According to a seventh aspect, this process is characterized in that, in order to select a plant having a modified starch content in the seed, the identity of the base or of a sequence of bases present at at least one nucleotide position of said nucleic acid corresponding to at least one of the nucleotides at position −830 to −824, −362, −347, −296, −15, +515, +587, +1068, +1505 and +2939 of the Sh2 gene of sequence SEQ ID No. 1 is determined.

According to an eighth aspect, this process is characterized in that, in order to select a plant having a modified amylose content in the seeds, the identity of the base or of a sequence of bases present at at least one nucleotide position of said nucleic acid corresponding to at least one of the nucleotides at position −438, −266, +678, +960, −921, −580 to −573, −277, +35, +304, +1059, +1081, +1867, +2514, +2771 and +3123 of the Sh2 gene of sequence SEQ ID No. 1 is determined.

According to a ninth aspect, this process is characterized in that, in order to select a plant having a modified protein/starch ratio in the seed, the identity of the base or of a sequence of bases present at at least one nucleotide position of said nucleic acid corresponding to at least one of the nucleotides at position −168, +1473, +1542, +2983, −830 to −824, −362, −347, −296, −15, +515, +587, +1068, +1505 and +2939 of the Sh2 gene of sequence SEQ ID No. 1 is determined.

According to a tenth aspect, said process is characterized in that it is carried out on the DNA taken from plants at the plantlet stage and/or at the early stage and/or at the vegetative stage.

Preferably, the plant is a cereal, preferably a straw cereal.

Preferably, the plant is chosen from maize and sorghum.

Nucleotide Probes and Primers

The invention also relates to the probes and the primers obtained from the nucleotide sequences described above which are specific for an allele of the Sh2 gene. One of the applications of the invention consists in using these probes and/or primers for detecting the polymorphism of the Sh2 gene at at least one of the polymorphic sites such as they have been defined above. The probes and the primers obtained according to one of the aspects of the invention can be used as markers specific for an allele of the Sh2 gene. The allele-specific nucleotide primers, or primers which make it possible to discriminate according to known alleles, consist preferably of 8 to 40 nucleotides, more precisely of 17 to 25 nucleotides. The preparation of such primers is known to those skilled in the art. In general, such primers comprise a sequence which is completely complementary to the sequence defining the reference allele or the “variant” allele associated with an improved phenotypic seed quality characteristic, as defined previously in the description. The primers, which are subjects of the invention, thus prepared carry one or more forms of labeling in order to facilitate their detection. For example, the labeling may be fluorimetric (digoxigenin, fluorescein, etc.), radioactive (for example ³²P), enzymatic (peroxydase, alkaline phosphatase, etc.), or produced by microbeads of gold, of colored glass or of plastic.

The nucleic acids according to the invention, and in particular the nucleotide sequences SEQ ID No. 2 to SEQ ID No. 39, and also the nucleic acids of sequences complementary to the sequences SEQ ID No. 2 to SEQ ID No. 39, are useful for producing probes or primers which make it possible, when they are used in hybridization, extension or amplification reactions, to distinguish between the two alleles of a given polymorphic site of the Sh2 gene characterized according to the invention.

Also part of the invention are the nucleotide probes and primers which hybridize with a nucleic acid chosen from the sequences SEQ ID No. 2 to SEQ ID No. 39, or with a nucleic acid of sequence complementary to one of the sequences SEQ ID No. 2 to SEQ ID No. 39.

A subject of the invention is also a nucleotide probe or a nucleotide primer, characterized in that it makes it possible to distinguish between the various alleles of a polymorphic site at at least one of the positions −921, −830 to −824, −580 to −573, −438, −362, −347, −296, −277, −266, −168, −15, +35, +304, +515, +587, +678, +960, +1059, +1068, +1081, +1473, +1505, +1542, +1867, +2514, +2771, +2939, +2983 and +3123 of the Sh2 gene of sequence SEQ ID No. 1.

The invention also relates to the use of a probe or of a primer as defined above, as a marker for at least one polymorphic site of the Sh2 gene.

The invention also relates to the diagnostic kits comprising nucleotide sequences as defined above, specific for the favorable allele of the Sh2 gene for one or more of the agronomic characteristics chosen from the number of grains per ear, the mass of the mature grain, and the starch content, amylose content or protein content of the grain.

A subject of the invention is also a set or kit to predict the phenotypic plant seed quality characteristics, characterized in that it comprises:
a) a probe or a plurality of probes or primers as defined above;
b) where appropriate, the reagents required to carry out a hybridization or amplification reaction.

According to a first aspect, the detection pack or kit is characterized in that the probe(s) is (are) immobilized on a support.

According to a second aspect, the detection pack or kit is characterized in that the oligonucleotide probes comprise a detectable label.

A nucleotide primer or a nucleotide probe according to the invention can be prepared by any suitable method well known to those skilled in the art, including by cloning and the action of restriction enzymes or else by direct chemical synthesis according to techniques such as the phosphodiester method of Narang et al. (1979) or of Brown et al. (1979), the diethylphosphoramidite method of Beaucage et al. (1980) or else the solid support technique described in European patent No. EP-0.707.592. Each of the nucleic acids according to the invention, including the oligonucleotide probes and primers described above, can be labeled, if desired, by incorporating a detectable molecule, i.e. a label detectable by spectroscopic, photochemical, biochemical, immunochemical or else chemical means.

For example, such labels may consist of radioactive isotopes (³²P, ³H, ³⁵S), fluorescent molecules (5-bromodeoxyuridine, fluorescein, acetylaminofluorene) or else ligands such as biotin.

The probes are preferably labeled by incorporation of labeled molecules into the polynucleotides by primer extension, or else by addition to the 5′ or 3′ ends.

Examples of nonradioactive labeling of nucleic acid fragments are described in particular in French patent No. FR-78.10975 or else in the articles by Urdea et al. (1988) or Sanchez Pescador et al. (1988).

Advantageously, the probes according to the invention can have structural characteristics such that they allow amplification of the signal, such as the probes described by Urdea et al; (1991) or else in European patent No. EP-0.225.807 (Chiron).

The oligonucleotide probes according to the invention can be used in particular in Southern hybridizations to the DNA of the Sh2 gene or else in hybridizations to the messenger RNA of this gene when expression of the corresponding transcript is sought in a sample.

The probes according to the invention can also be used for detecting PCR amplification products or else for detecting mismatches.

Nucleotide probes or primers according to the invention can be immobilized on a solid support. Such solid supports are well known to those skilled in the art and encompass the surfaces of microtitration plate wells, polystyrene beads, magnetic beads, nitrocellulose strips or else microparticles such as latex particles.

Means of Expression of the Nucleic Acids Comprising a Polymorphic Site of the Sh2 Gene

The invention also relates to the use of the methods described above for isolating and/or cloning the particular and/or favorable allelic forms of the Sh2 gene, in particular in combination with molecular biology techniques such as, for example, PCR, RT-PCR or allele sequencing (for example pyrosequencing, etc). Those skilled in the art may find the description of the main cloning techniques in general works such as “Guide to Molecular Cloning Techniques” (Berger and Kimmel, ref), “Molecular Cloning A—laboratory manual” (Sambrook et al., 2001), “Current protocols in molecular biology” (Ausubel et al., 1997).

A nucleic acid comprising the complete sequence of a given allele of the Sh2 gene and comprising at least one polymorphic base of a polymorphic site as defined in the present description, or a nucleic acid comprising all the exons of this given allele of the Sh2 gene, can be obtained according to techniques well known to those skilled in the art, such as, for example, by the technique of PCR amplification using primers which hybridize specifically with the chosen target nucleotide ends of the Sh2 gene, as is in particular described in the examples.

In particular, those skilled in the art may use the sequence SEQ ID No. 1 as a basis for synthesizing nucleotide probes and nucleotide primers for isolating and cloning any allelic nucleic acid of the Sh2 gene.

For example, those skilled in the art can obtain a nucleic acid derived from the Sh2 gene and comprising at least one allele associated with an improved phenotypic characteristic of seed quantity or quality, of at least one polymorphic site according to the invention, by amplifying respectively the 5′ portion and the 3′ portion of the Sh2 gene using the nucleotide sequence SEQ ID No. 1 as a basis.

A subject of the invention is also a recombinant vector into which has been inserted a nucleic acid comprising the complete sequence of a given allele of the Sh2 gene and comprising at least one polymorphic base of a polymorphic site as defined in the present description, or a nucleic acid comprising all the exons of this given allele of the Sh2 gene.

Preferably, such a nucleic acid is a nucleic acid derived from the Sh2 gene of sequence SEQ ID No. 1 and comprising at least one polymorphic base or at least one polymorphic nucleotide sequence at at least one of the positions −921, −830 to −824, −580 to −573, −438, −362, −347, −296, −277, −266, −168, −15, +35, +304, +515, +587, +678, +960, +1059, +1068, +1081, +1473, +1505, +1542, +1867, +2514, +2771, +2939, +2983 and +3123 of the Sh2 gene of sequence SEQ ID No. 1.

A first nucleic acid which is a subject of the invention is a nucleic acid capable of conferring on a plant, preferably a cereal, in particular on a maize, a modified number of seeds compared to the reference maize of the variety Black Mexican Sweet described by Shaw and Hannah (1992), said nucleic acid comprising the allelic form associated with the expression of the modified phenotypic seed quality characteristic, as defined in the present description, at at least one polymorphic site chosen from the polymorphic sites −168, +1473, +1542 and +2983 of the Sh2 gene of sequence SEQ ID No. 1.

A second nucleic acid which is a subject of the invention is a nucleic acid capable of conferring on a plant, preferably a cereal, in particular on a maize, a modified seed mass compared to the reference maize of the variety Black Mexican Sweet described by Shaw and Hannah (1992), said nucleic acid comprising the allelic form associated with the expression of the modified phenotypic seed quality characteristic, as defined in the present description, at at least one polymorphic site chosen from the polymorphic sites −168, +1473, +1542 and +2983 of the Sh2 gene of sequence SEQ ID No. 1.

A third nucleic acid which is a subject of the invention is a nucleic acid capable of conferring on a plant, preferably a cereal, in particular on a maize, a modified protein content in the seeds compared to the reference maize of the variety Black Mexican Sweet described by Shaw and Hannah (1992), said nucleic acid comprising the allelic form associated with the expression of the modified phenotypic seed quality characteristic, as defined in the present description, at at least one polymorphic site chosen from the polymorphic sites −168, +1473, +1542, +2983, −830 to −824, −362, −347, −296, −15, +515, +1068, +1505 and +2939 of the Sh2 gene of sequence SEQ ID No. 1.

A fourth nucleic acid which is a subject of the invention is a nucleic acid capable of conferring on a plant, preferably a cereal, in particular on a maize, a modified starch content in the seeds compared to the reference maize of the variety Black Mexican Sweet described by Shaw and Hannah (1992), said nucleic acid comprising the allelic form associated with the expression of the modified phenotypic seed quality characteristic, as defined in the present description, at at least one polymorphic site chosen from the polymorphic sites −830 to −824, −362, −347, −296, −15, +515, +587, +1068, +1505 and +2939 of the Sh2 gene of sequence SEQ ID No. 1.

A fifth nucleic acid which is a subject of the invention is a nucleic acid capable of conferring on a plant, preferably a cereal, in particular on a maize, a modified amylose content in the seeds compared to the reference maize of the variety Black Mexican Sweet described by Shaw and Hannah (1992), said nucleic acid comprising the allelic form associated with the expression of the modified phenotypic seed quality characteristic, as defined in the present description, at at least one polymorphic site chosen from the polymorphic sites −438, −266, +678, +960, −921, −580 to −573, −277, +35, +304, +1059, +1081, +1867, +2514, +2771 and +3123 of the Sh2 gene of sequence SEQ ID No. 1.

A sixth nucleic acid which is a subject of the invention is a nucleic acid capable of conferring on a plant, preferably a cereal, in particular on a maize, a modified protein/starch ratio in the seed compared to the reference maize of the variety Black Mexican Sweet described by Shaw and Hannah (1992), said nucleic acid comprising the allelic form associated with the expression of the modified phenotypic seed quality characteristic, as defined in the present description, at at least one polymorphic site chosen from the polymorphic sites −168, +1473, +1542, +2983, −830 to −824, −362, −347, −296, −15, +515, +587, +1068, +1505 and +2939 of the Sh2 gene of sequence SEQ ID No. 1.

The maize variety Black Mexican Sweet described by Shaw and Hannah (1992), which is the reference variety, can also be referred to as “wild-type” maize variety, for the purposes of the present description.

The techniques for measuring the number of seeds, the mass of the seed, the protein content of the seed, the starch content in the seed and the amylose content in the seed and calculation of the protein content/starch content ratio are part of the general technical knowledge of those skilled in the art.

The invention also relates to a recombinant vector, for example a recombinant cloning vector or a recombinant expression vector, into which has been inserted a nucleic acid as defined above.

According to one of the applications of the invention, the nucleotide sequences constituting particular allelic forms of the Sh2 gene can be cloned and transferred into cells, in particular for producing transgenic plants. The nucleotide sequences of the loci selected are introduced into the plant cells either in culture or in plant organs such as, for example, the leaves, the stems, the seeds, the roots, etc. Natural or synthetic expression of these sequences can be produced by functionally linking these sequences of interest to a promoter, including the construct in a vector, and introducing the vector into a host cell. An endogenous promoter linked to the nucleotide sequence to be introduced can favorably be used. The vectors conventionally used comprise both transcription and translation initiating and terminating sequences, and promoters for regulating the expression of this particular nucleotide sequence. The vectors can also comprise expression cassettes with at least one independent terminator sequence, sequences for replication of the cassette in eukaryotes or prokaryotes or both (shuttle vectors) and a selection marker for at least one of the two prokaryotic or eukaryotic systems (Giliman and Smith, 1979; Roberts et al., 1987; Schneider et al., 1995; Sambrook et al., 2001; Ausubel et al., 1997).

Among the transcription terminators which can be used, mention may be made of the 35S polyA terminator of the cauliflower mosaic virus (Franck et al. 1980) or the NOS polyA terminator, which corresponds to the 3′ noncoding region of the nopaline synthase gene of the Ti plasmid of Agrobacterium tumefaciens nopaline-type strain.

Among the transcription promoters which can be used, mention may in particular be made of “constitutive” promoters, “inducible” promoters and also tissue-specific promoters. A constitutive promoter allows a strong expression of the transcript in all the tissues of the regenerated plant and will be active under most environmental conditions, and in all the steps of cell transformation and differentiation; for example, the 35S promoter or the double promoter pd35S of CaMV described in the article by Kay et al., (1987), or the rice actin promoter followed by the rice actin intron contained in the plasmid pAct1-F4 described by Mc Elroy et al., 1991; or else the maize ubiquitin-1 promoter (Christensen et al., 1996). Alternatively, it may be advantageous to use an inducible transcription promoter sequence capable of being controlled by the environmental conditions or the developmental stage, such as, for example, the phenylalanine ammonia lyase (PAL), HMG-CoA reductase (HMG), chitinase, glucanase, proteinase inhibitor (PI), PR1 family gene, nopaline synthase (nos) or vspB gene (U.S. Pat. No. 5,670,349) promoters, all these promoters being recalled with the references of the corresponding publications in Table 3 of U.S. Pat. No. 5,670,349. Another category of promoters which can be used includes tissue-specific promoters, such as, for example, the seed tissue-specific promoters (Datla, R. et al., 1997), in particular the napine (EP-0.255.378), glutenin, helianthinin (WO-92/17580), albumin (WO-98/45460), oleosin (WO-98/45461), ATS1 or ATS3 (WO-99/20775) promoters.

The most widely used methods for introducing nucleic acids into bacterial cells can be used in the context of this invention. This may be the fusion of recipient cells with bacterial protoplasts containing the DNA, electroporation, projectile bombardment, infection with viral vectors, etc. Bacterial cells are often used to amplify the number of plasmids containing the construct comprising the nucleotide sequence which is the subject of the invention. The bacteria are placed in culture and the plasmids are then isolated according to methods well known to those skilled in the art (reference should be made to the protocol manuals already mentioned), including the plasmid purification kits sold commercially, such as, for example, EasyPrepI from Pharmacia Biotech or QIAexpress Expression System from Qiagen. The plasmids thus isolated and purified are then manipulated so as to produce other plasmids which will be used to transfect the plant cells.

The transformation of plant cells can be carried out by various methods, such as, for example, transfer of the abovementioned vectors into plant protoplasts after incubation of the latter in a solution of polyethylene glycol in the presence of divalent cations (Ca 2+), electroporation (Fromm et al. 1985), the use of a particle gun, or cytoplasmic or nuclear microinjection (Neuhaus et al, 1987).

One of the methods for transforming plant cells which can be used in the context of the invention is infection of the plant cells with a bacterial cellular host comprising the vector containing the sequence of interest. The cellular host can be Agrobacterium tumefaciens (An et al. 1986), or A. rhizogenes (Guerche et al. 1987).

Preferably, the transformation of the plant cells is carried out by transfer of the T region of the tumor-inducing extrachromosomal circular plasmid Ti of A. tumefaciens, using a binary system (Watson et al., 1994). To do this, two vectors are constructed. In one of these vectors, the T-DNA region has been eliminated by deletion, with the exception of the right and left edges, a marker gene being inserted between them so as to allow selection in the plant cells. The other partner of the binary system is an auxiliary Ti plasmid, which is a modified plasmid no longer having any T-DNA but still containing the vir virulence genes necessary for transforming the plant cell. This plasmid is maintained in Agrobacterium.

According to a preferred embodiment, the method described by Ishida et al. (1996) can be applied for the transformation of monocotyledons, in particular maize. According to another protocol, the transformation is carried out according to the method described by Finer et al. (1992) using a tungsten or gold particle gun.

A subject of the invention is also the use of a nucleic acid derived from the Sh2 gene of sequence SEQ ID No. 1 as defined above, for transforming a host cell.

Preferably, the host cell is a bacterial host cell, for example an Agrobacterium tumefaciens cell or a plant cell, preferably a cereal cell, and entirely preferably a maize, rice, wheat, rye or barley cell.

The invention also relates to the cells transformed with the nucleotide sequences constituting particular allelic forms of the Sh2 gene, including the microorganisms (viruses and bacteria) and the plant cells, in particular maize cells.

It relates in particular to a host cell transformed with a nucleic acid derived from the Sh2 gene or with a recombinant vector, as defined above.

The invention also relates to the plants regenerated from the transformed plant cells described above, and also to the plants for which at least one of the parents has been regenerated from a transformed plant cell comprising at least one of the nucleotide sequences constituting a particular allelic form of the Sh2 gene.

The invention also relates to the use of a nucleic acid, of a recombinant vector or of a transformed host cell, defined according to the invention, for producing a transformed plant capable of producing seeds with improved industrial or agrofoods quality.

Also falling within the context of the invention is a transformed plant comprising a plurality of transformed host cells according to the invention.

A subject of the invention is also a process for obtaining a transformed plant capable of producing seeds with improved industrial or agrofoods qualities, characterized in that it comprises the following steps:
a) transforming at least one plant cell with a nucleic acid or with a recombinant vector according to the invention;
b) selecting the transformed cells obtained in step a) which have integrated into their genome at least one copy of a nucleic acid according to the invention;
c) regenerating a transformed plant from the transformed cells obtained in step b).

The invention extends to a transformed plant or any part of a transformed plant as defined in the present description, such as the root, but also the aereal parts such as the stem, the leaf, the flower and especially the seed. A subject of the invention is also a plant seed or grain produced by a transformed plant as defined above. Typically, such a transformed seed or such a transformed grain comprises one or more cells comprising in their genome one or more copies of a nucleic acid defined according to the invention, where appropriate in a controlled and inducible manner.

Also part of the invention is any product of transformation of a seed as defined in the present description.

Antibodies Directed Specifically Against the SH2 Proteins Produced by a Nucleic Acid Comprising a Site for which the Polymorphism is Described According to the Invention

The invention also relates to the monoclonal or polyclonal antibodies which recognize specifically polypeptides corresponding to alleles of the Sh2 gene, these alleles comprising at least one of the forms described at positions No. −921, −830 to −824, −580 to −573, −438, −362, −347, −296, −277, −266, −168, −15, 35, 304, 515, 587, 678, 960, 1059, 1068, 1081, 1473, 1505, 1542, 1867, 2514, 2771, 2939, 2983, 3123.

Antibodies preferred according to the invention are the following antibodies:
the antibodies which recognize specifically the amino acid region, of an SH2 polypeptide, encoded by the nucleotides located in the vicinity of the polymorphic site +678, these antibodies being capable of discriminating between the presence of an alanine residue and the presence of a threonine residue encoded by the codon comprising the polymorphic base of this polymorphic site; and
the antibodies which recognize specifically the amino acid region, of an SH2 polypeptide, encoded by the nucleotides located in the vicinity of the polymorphic site +2983, these antibodies being capable of discriminating between the presence of a leucine residue and the presence of a serine residue encoded by the codon comprising the polymorphic base of this polymorphic site.

The invention also relates to the diagnostic kit comprising a mixture of these antibodies.

The antibodies defined above are useful in particular for predicting the phenotypic characteristics of seed quantity or quality, without requiring long and expensive direct analysis, for example biochemical analysis, of these phenotypic characteristics.

Antibodies against the SH2 polypeptides defined above can be prepared according to the conventional techniques well known to those skilled in the art.

For the purpose of the present invention, the term “antibodies” will be intended in particular to mean polyclonal or monoclonal antibodies or fragments (for example F(ab)′₂, F(ab) fragments) or else any polypeptide comprising a domain of the initial antibody which recognizes the target polypeptide or polypeptide fragment according to the invention.

Monoclonal antibodies can be prepared from hybridomas according to the technique described by Kohler and Milstein (1975).

The present invention also relates to antibodies directed against a polypeptide as described above, or a fragment or a variant of said polypeptide, as produced in the trioma technique or else the hybridoma technique described by Kozbor et al. (1983).

The invention also relates to single-chain Fv (ScFv) antibody fragments as described in U.S. Pat. No. 4,946,778 or else by Martineau et al. (1998).

The antibodies according to the invention also comprise antibody fragments obtained using phage libraries as described by Ridder et al. (1995) or else humanized antibodies as described by Reinmann et al. (1997) and Leger et al. (1997). The preparations of antibodies according to the invention are useful in immunodetection assays intended to identify the presence and/or the amount of an SH2 polypeptide as defined above or of a peptide fragment thereof, present in a sample.

An antibody according to the invention may also comprise an isotopic or nonisotopic detectable label, for example a fluorescent label, or else may be coupled to a molecule such as biotin, according to techniques well known to those skilled in the art.

Thus, a subject of the invention is also a process for detecting the presence of a polypeptide in accordance with the invention in a sample, said process comprising the steps of:
a) bringing the sample to be tested into contact with an antibody as described above;
b) detecting the antigen/antibody complex formed.

The invention also relates to a diagnostic pack or kit for detecting the presence of a polypeptide in accordance with the invention in a sample, said pack comprising:
a) an antibody as defined above;
b) where appropriate, one or more reagents required for the detection of the antigen/antibody complex formed.

In addition, a subject of the invention is also a database comprising at least any one of the nucleotide sequences described, obtained, isolated or identified in at least one of the applications of the invention.

The present invention is also illustrated, without however being limited, by the following figures and examples.

### EXAMPLES

### Example 1

### Identification of Polymorphic Sites in the Sh2 Gene

1.1 Plant Material

In total, 33 inbred maize lines were analyzed for, firstly, sequencing of the Sh2 gene and, secondly, grain phenotype measurements. These lines are of European, American or tropical origin, and, on the RFLP database, they are poorly related (Dubreuil et al. 1996, and unpublished data). The grain characteristics of lines A to Z and a to e are shown in Table 1.

TABLE 1
Characteristics of the lines

| Lines | Grain |
|---|---|
| A | Flint-Dent |
| B | Dent |
| C | Flint-Dent |
| D | Flint |
| E | Dent |
| F | Flint |
| G | Dent |
| H | Flint |
| I | Dent |
| J | Dent |
| K | Flint-Dent |
| L | Dent |
| M | Dent |
| N | Dent |
| O | Dent |
| P | Dent |
| Q | Dent |
| R | Dent |
| S | Dent |
| T | Dent |
| U | Dent |
| V | Floury |
| W | Dent |
| X | Dent |
| Y | Dent |
| Z | Flint |
| a | Flint-Dent |
| b | Dent |
| c | Flint |
| d | Flint |
| e | Flint |
| f | Flint |

1.2 Detection of the Polymorphism of the Sh2 Gene

The DNA was isolated from young leaves by a method described by Causse et al. (1995). Two overlapping fragments were amplified by PCR using primers designed from the reference sequence of the Sh2 gene in the line Black Mexican Sweet, SEQ ID No. 1 (Shaw and Hannah, 1992). The first fragment (Sh2-I), in the region of 2653 bp in length, covers approximately 1000 bp upstream of the supposed TATA box, the first three introns, the first three exons and a portion of exon 4. The second fragment (SH2-II) is in the region of 2455 bp in length and covers introns 3 to 12, exons 3 to 12 and approximately 30 bases of exon 13. The primers used are as follows (position on the reference sequence): for Sh2-I:

(SEQ ID No. 40)

5′-CTGGGCAGGGAGAGCTAT (position −1008)

(SEQ ID No. 41)

3′-GGATATCAATAAGCCTGTAACAT (position 1623)

for Sh2-II:

(SEQ ID No. 42)

5′-TGCAGCATTCTCAAACACAG (position 1197)

(SEQ ID No. 43)

3′-CGATGTTGCATTCTCTCAGAA (position 3630)

For the two fragments and all the lines, the PCR amplifications were carried out in 100 μl (approximately 0.1 to 1.5 μg of DNA, 1.75 U of taq polymerase Roche Expand High Fidelity, 0.5 μM of each primer, 0.3 μM of dNTP, 1.875 mM of Mg²⁺) under the following conditions:
denaturation at 94° C.: 2 min
9 cycles:
   denaturation at 94° C.: 30 s
   hybridization from 60 to 52° C. (decrease of 1° C. per cycle): 30 s
   elongation at 72° C.: 2 min
25 cycles:
   denaturation at 94° C.: 30 s
   hybridization at 51° C.: 30 s
   elongation at 72° C.: 2 to 10 min (increase of 20 s per cycle)
elongation at 72° C.: 7 min.

After purification on gel by means of the QIAEX kit from Quiagen, the PCR products were sequenced by the companies Genome Express or Genaxis. First of all, the PCR primers were used for the sequence, and then the central portion of the fragments was sequenced using the following new primers:

For Sh2-I:

(SEQ ID No. 44)

5′-ATGTCCTGCACCTAGGGAGC (position −424),

(SEQ ID No. 45)

5′-CCACCAGTATGCCCTCCTCA (position −58),

(SEQ ID No. 46)

3′-GACCCTATTTGAACAAATCTT (position 852),

(SEQ ID No. 47)

3′-GCAGCAACTCTAAGGTCTATTT (position 961),

(SEQ ID No. 48)

5′-TTTGGGAGACTTCCAGTCAA (position 1503),

For Sh2-II:

(SEQ ID No. 49)

3′-CATCGTCCTCGACATGTTT (position 2365),

(SEQ ID No. 50)

3′-GGAAAGCAGATTAGACCATAT (position 2486),

(SEQ ID No. 51)

3′-TGGAAACTGGAAACAAAAACAAC (position 3098)

A first correction of the sequences, the contigs and the sequence alignment was carried out using the Sequencing Analysis and Sequence Navigator programs from ABI. The sequences were aligned either visually or using the Clustal multiple alignment procedure of the Sequence Navigator program. The fragments were only sequenced on a single strand. However, verifications were performed. First of all, the overlapping portion of the sequence fragments never showed any incoherence during the production of the contigs. In addition, the PCR amplification and the sequencing were repeated on a total of approximately 10 000 bp in order to verify all the singleton polymorphisms (the case when a single line differs from all the others). Identical results were always obtained for the two repetitions.

The sites for which differences were observed among the Sh2 sequences are identified by their position with respect to the reference sequence of the line Black Mexican Sweet SEQ ID No. 1 (Shaw and Hannah, 1992). Over the entire region sequenced, 72 polymorphic sites were observed. Among these polymorphisms, 19 show a difference for a single line, all the others being identical. It will not be possible to use these singletons in searching for associations with phenotypic grain variability. Among the remaining 53 informative sites, some are completely redundant with respect to one another, i.e. they provide identical information with regard to the similarities between lines and therefore allow identical discrimination in two groups of lines. Among the 20 informative and nonredundant sites or groups of sites, those found to be statistically associated with phenotypic measurements are indicated in the detailed presentation of the invention. Table 2 gives the allelic form of each line, or group of lines, for these polymorphisms.

Table 2: Allelic forms for the 29 polymorphisms described, including 20 SNP and 9 indels, named by virtue of their position on the reference sequence SEQ ID No. 1 of the line Black Mexican Sweet (BMS). The notations ¹ to ⁴ indicate the redundant sites: the site −168 is redundant with the sites 1473, 1542 and 2983; the indel −830 to −824 is redundant with the sites −362, −347, −296, −15, 515, 587, 1068, 1505 and 2939; the site −438 is redundant with the sites −266, 678 and 960; the site −921 is redundant with the sites −580 to −573, −277, 35, 304, 1059, 1081, 1867, 2514, 2771 and 3123. The haplotypes H1 to H6 correspond to the following lines:

| | | −830 to | −580 to | | | | | | | | | | | | |
|---|---|---|---|---|---|---|---|---|---|---|---|---|---|---|---|
| | −921¹ | −824² | −573¹ | −438³ | −362² | −347² | −296² | −277¹ | −266³ | −168⁴ | −15² | 35¹ | 304¹ | 515² | 587² |
| sites | SNP | Indel | indel | SNP | SNP | SNP | SNP | SNP | SNP | SNP | SNP | SNP | indel | SNP | SNP |
| H1 | A | TGAGA | TCACCTAT | A | G | C | C | C | T | G | G | C | . | T | T |
| | | AA | | | | | | | | | | | | | |
| H2 | G | . | . | . | . | . | . | T | . | . | . | T | T | . | . |
| H3 | G | . | . | G | . | . | . | T | C | . | . | T | T | . | . |
| H4 | G | . | . | . | A | T | T | T | . | . | A | T | T | C | C. |
| H5 | G | . | . | . | . | . | . | T | . | A | . | T | T | . | . |

| | 678³ | 960³ | 1059¹ | 1068² | 1081¹ | 1473⁴ | 1505² | 1542⁴ | 1867¹ | 2514¹ | 2771¹ | 2939² | 3983⁴ | 3123 |
|---|---|---|---|---|---|---|---|---|---|---|---|---|---|---|
| sites | SNP | SNP | SNP | SNP | indel | SNP | indel | indel | SNP | indel | indel | SNP | SNP | indel |
| H1 | G | G | C | T | A | T | . | . | T | T | . | T | T | . |
| H2 | . | . | G | . | . | . | . | . | C | . | T | . | . | GTTTTTATTTA |
| H3 | A | A | G | . | . | . | . | . | C | . | T | . | . | GTTTTTATTTA |
| H4 | . | . | G | G | . | . | T | . | C | . | T | G | . | GTTTTTATTTA |
| H5 | . | . | G | . | . | C | . | T | C | . | T | . | C | GTTTTTATTTA |

H1: BMS, E, c, M, Q, F, G
H2: A, D, L, P, R, b, H, O, S, T, a, U, X, N
H3: B, K, Y
H4: J, V, I
H5: W, C, Z

### Example 2

### Identification of the Statistically Significant Associations Between a Given Allele of the Polymorphic Sites of the Sh2 Gene and One or More Phenotypic Seed Quality Characteristics

2.1 Phenotypic Grain Measurements

Three experiments in the field were carried out. The first year (year 1), 30 lines were studied, i.e. the lines shown in Table 1, with the exception of the lines RX01, RX02 and RX03. For the experiments set up in years 2 and 3, the 33 lines were randomized and repeated in three blocks. Each line was represented, in each block, by one to four plants sown side by side. For each of the three experiments, the plants were self-pollinated and the grains harvested at maturity. The analyses of associations with the molecular polymorphisms of Sh2 related to average values for each characteristic per experiment.

The protein, starch and amylose contents of each line were assayed manually for the year-1 experiment (see detail of the methods in Sene et al., 2000) and predicted by near-infrared spectrometry (NIRS) at the CIRAD-Montpellier by C. Mestres and F. Davrieux for year-2 and year-3 experiments. The acquisition of the spectra was carried out on whole grains using a Nirsystem 6500 device. The spectral data, for a wavelength range of 400 to 2500 nm, were collected and analyzed using the NIRS 2 software, version 4.0 (Infrasoft International). Since the range of predicted values was not completely superimposable on the range of values which enabled the equations to be calibrated, about thirty samples derived from our two experiments were used to refine the calibration.

2.2 Associations Between Polymorphisms of the Sh2 Gene and Phenotypic Characteristics of Interest

The polymorphisms described above are statistically associated with grain characteristics such as the number of grains per ear, the mass of the mature grain, the protein, starch and amylose content of the grain, and the protein content to starch content ratio in the grain. These associations were demonstrated by multiple regression tests using the SAS program (1990). When a characteristic is significantly associated with several sites, it is possible to define the share of variability of the characteristic, explained by all the sites involved (R²_{T}, last column of Table 3). For example, it may be noted that, in the first-year experiment, the combination of the indel −830 to −824 and of the SNP −168 makes it possible to explain 64% of the variance of the protein/starch ratio among all the lines. The redundancy of the sites (see Table 2) makes the fine interpretation of causality between a site and a characteristic difficult. In addition, it is possible that polymorphisms not described in the invention and present in the vicinity of the sequenced region, either in the promoter region or in the unsequenced 3′ portion of the gene, are also redundant with the sites described and therefore associated with the same characteristics. In the potato, an important region for regulating expression of the gene is found more than 2 kb upstream of the TATA box (Muller-Rober et al., 1994). However, whether or not the relationship of causality between molecular and phenotypic polymorphism is known, the statistical associations described in the invention make it possible to use the SNPs and the indels described for the purposes of phenotypic prediction or of improving the genetic value of maize seeds.

Table 3: Results of the tests for associations by multiple regressions between each characteristic of the grain and the polymorphic sites in the sequence of Sh2 for 25 to 33 lines. The sites indicated are completely redundant with other sites along the sequence of Sh2 (see Table 2). The columns n1, ave1, n2, and ave2 contain the numbers and the average values for the characteristic for the two groups of lines discriminated by the corresponding polymorphic sites. F: Fisher test value, P: degree of significance, R²: share of phenotypic variation explained by a site, R²_{T}: share of phenotypic variation explained by a group of sites combined by multiple regression.

TABLE 3

| Characteristic | Experiment | sites | n1 | ave 1 | n2 | ave 2 | F | P | R² | R²_{T} |
|---|---|---|---|---|---|---|---|---|---|---|
| number of grains | year 1 | −168 | 30 | 141.6 | 3 | 221.2 | 9.94 | 0.004 | 0.24 | |
| number of grains | year 3 | −168 | 30 | 125.9 | 3 | 244.0 | 11.73 | 0.002 | 0.27 | |
| grain mass | year 1 | −168 | 24 | 225.1 | 3 | 289.0 | 9.37 | 0.005 | 0.27 | |
| protein content | year 1 | −830 to −824 | 22 | 11.97 | 3 | 15.64 | 15.05 | 0.001 | 0.40 | |
| protein content | year 2 | −830 to −824 | 30 | 13.17 | 3 | 15.41 | 5.96 | 0.021 | 0.16 | |
| protein content | year 3 | −830 to −824 | 29 | 13.41 | 2 | 15.80 | 8.50 | 0.007 | 0.23 | 0.33 |
| protein content | year 3 | −168 | 28 | 13.71 | 3 | 12.19 | 4.46 | 0.043 | 0.11 | |
| starch content | year 2 | −830 to −824 | 30 | 72.85 | 3 | 66.85 | 6.52 | 0.016 | 0.17 | |
| amylose content | year 1 | −921 | 22 | 17.6 | 5 | 22.7 | 4.60 | 0.042 | 0.16 | |
| amylose content | year 2 | −438 | 30 | 23.42 | 3 | 46.45 | 11.20 | 0.002 | 0.26 | |
| protein/starch | year 1 | −830 to −824 | 22 | 0.149 | 3 | 0.218 | 28.22 | 0.000 | 0.55 | 0.64 |
| protein/starch | year 1 | −168 | 23 | 0.160 | 2 | 0.119 | 3.75 | 0.003 | 0.09 | |
| protein/starch | year 2 | −830 to −824 | 30 | 0.194 | 3 | 0.229 | 5.68 | 0.024 | 0.15 | 0.26 |
| protein/starch | year 2 | −168 | 30 | 0.201 | 3 | 0.168 | 4.79 | 0.036 | 0.11 | |
| protein/starch | year 3 | −830 to −824 | 29 | 0.197 | 2 | 0.236 | 8.15 | 0.008 | 0.22 | 0.35 |
| protein/starch | year 3 | −168 | 28 | 0.202 | 3 | 0.175 | 5.30 | 0.029 | 0.13 | |

### REFERENCES

An et al., (1986) Plant Physiol. 81, 86-91
Ausubel et al., (1997) Current protocols in molecular biology
Bae et al. (1990) Maydica 35: 317-322.
Beaucage et al. (1981), Tetrahedron Lett., 22:1859-1862.
Berger et Kimmel, Guide to Molecular Cloning Techniques
Bhave et al. (1990) The Plant Cell 2: 581-588.
Bourdon et al., (2001) EMBO reports 2 (5) 394-398.
Brown et al. (1979), Methods Enzymol., 68:109-151.
Causse et al. (1995) Molecular Breeding 1: 259-272.
Christensen et al. (1996), Transgenic. Res., 5:213
Datla, R et al. (1997), Biotechnology Ann. Rev., 3:269-296
Dellaporta S. L., Wood J. and Hicks J. B, (1983), Plant Mol. Biol. Reporter 1(4), 19-21.
Dubreuil et al. (1996) Crop Sci. 36: 790-799.
Finer et al. (1992) Plant Cell Report, 11, 323-328
Franck et al., (1980) Cell, 21, 285-294
Fromm et al., (1985) Proc. Nat. Acad. Sci. 82, 5824
Gibbs et al. (1989), Nucleic acids research, 17, 2347.
Giliman et Smith (1979), Gene 8, 81
Giroux et al., (1996), Proc. Natl. Acad. Sci. USA 93: 5824-5829.
Goldman et al. (1993) Theor. Appl. Genet. 87: 217-224.
Guerche et al. (1987), Mol Gen. Genet 206, 382
Ishida et al. (1996) Nature biotechnology 14, 745-750
Kay et al., (1987) Science 236, 4805
Kohler G and Milstein C;, (1975), Nature, volume 256:495
Kozbor et al., (1983), Hybridoma, vol. 2 (1):7-16.
Leger O J et al., (1997), Hum Antibodies, vol. 8 (1):3-16
Lewin, (1999), Genes VII
Martineau P et al., (1998), J. Mol. Biol. vol. 280(1):117-127.
Muller-Rober et al. (1994), The plant cell 6, 601-612.
Neuhaus et al., (1987). Theor. Appl. Genet. 75(1), 30-36
Nyrèn et al. (1997), Anal. Biochem, 244, 367-373
Reinmann K A et al. (1997), Aids Res. Hum retroviruses, vol. 13 (11):933-943.
Ridder R. et al., (1995), Biotechnology (NY), vol. 13 (3):255-260.
Roberts et al. (1987), Nature 328, 731
Sambrook et al., (2001), Molecular Cloning A—laboratory manual
Sanchez Pescador, (1988), J. Clin. Microbiol., 26 (10):1934-1938.
SAS (1990) SAS user guide: statistics. Version 6. SAS institute, Cary, N.C.
Schneider et al. (1995), Protein expr. Purif. 6435, 10
Séne et al. (2000) Plant Physiol. Biochem. 38: 459-472.
Shaw and Hannah (1992), Plant Physiol 98, 1214-1216
Urdea et al. (1988), Nucleic Acids Research, 11:4937-4957.
Watson et al. (1994) ADN recombinant [Recombinant DNA], Ed. De Boek University, 273-292

1. A process for selecting plants having improved phenotypic seed quality characteristics, which comprises detecting a polymorphic base or a polymorphic nucleotide sequence with a nucleotide probe or a nucleotide primer;
wherein the polymorphic base or polymorphic nucleotide sequence defines an allele of a polymorphic site of the Sh2 gene of sequence SEQ ID No. 1, said polymorphic base or said polymorphic nucleotide sequence being contained in a nucleic acid included in an Sh2 gene, said nucleic acid being a member selected from the group consisting of:
(a) a nucleic acid in which the nucleotide corresponding to the nucleotide at position −921 of the Sh2 gene is a G;
(b) a nucleic acid in which the nucleotides corresponding to the nucleotides at positions −830 to −824, of sequence 5′-TGAGAAA-3′, of the Sh2 gene are absent;
(c) a nucleic acid in which the nucleotides corresponding to the nucleotides at positions −580 to −573, of sequence 5′-TCACCTAT-3′, of the Sh2 gene are absent;
(d) a nucleic acid in which the nucleotide corresponding to the nucleotide at position −438 of the Sh2 gene is a G;
(e) a nucleic acid in which the nucleotide corresponding to the nucleotide at position −362 of the Sh2 gene is an A;
(f) a nucleic acid in which the nucleotide corresponding to the nucleotide at position −347 of the Sh2 gene is a T;
(g) a nucleic acid in which the nucleotide corresponding to the nucleotide at position −296 of the Sh2 gene is a T;
(h) a nucleic acid in which the nucleotide corresponding to the nucleotide at position −277 of the Sh2 gene is a T;
(i) a nucleic acid in which the nucleotide corresponding to the nucleotide at position −266 of the Sh2 gene is a C;
(j) a nucleic acid in which the nucleotide corresponding to the nucleotide at position −168 of the Sh2 gene is an A;
(k) a nucleic acid in which the nucleotide corresponding to the nucleotide at position −15 of the Sh2 gene is an A;
(l) a nucleic acid in which the nucleotide corresponding to the nucleotide at position +35 of the Sh2 gene is a T;
(m) a nucleic acid in which an additional T is found after the nucleotide at position +304 of the Sh2 gene;
(n) a nucleic acid in which the nucleotide corresponding to the nucleotide at position +515 of the Sh2 gene is a C;
(o) a nucleic acid in which the nucleotide corresponding to the nucleotide at position +587 of the Sh2 gene is a C;
(p) a nucleic acid in which the nucleotide corresponding to the nucleotide at position +678 of the Sh2 gene is an A;
(q) a nucleic acid in which the nucleotide corresponding to the nucleotide at position +960 of the Sh2 gene is an A;
(r) a nucleic acid in which the nucleotide corresponding to the nucleotide at position +1059 of the Sh2 gene is a G;
(s) a nucleic acid in which the nucleotide corresponding to the nucleotide at position +1068 of the Sh2 gene is a G;
(t) a nucleic acid in which the nucleotide A corresponding to the nucleotide at position +1081 of the Sh2 gene is absent;
(u) a nucleic acid in which the nucleotide corresponding to the nucleotide at position +1473 of the Sh2 gene is a C;
(v) a nucleic acid in which an additional T is present after the nucleotide at position +1505 of the Sh2 gene;
(w) a nucleic acid in which an additional T is present after the nucleotide at position +1542 of the Sh2 gene;
(x) a nucleic acid in which the nucleotide corresponding to the nucleotide at position +1867 of the Sh2 gene is a C;
(y) a nucleic acid in which the nucleotide T corresponding to the nucleotide at position +2514 of the Sh2 gene is absent;
(z) a nucleic acid in which an additional T is present after the nucleotide at position 2771 of the Sh2 gene;
(ab) a nucleic acid in which the nucleotide corresponding to the nucleotide at position +2939 of the Sh2 gene is a G;
(ac) a nucleic acid in which the nucleotide corresponding to the nucleotide at position +2983 of the Sh2 gene is a C; and
(ad) a nucleic acid comprising the insertion of the sequence 5′-GTTTTTATTTA-3′ after the nucleotide corresponding to the nucleotide at position +3123 of the Sh2 gene.

2. The process as claimed in claim 1, wherein the nucleotide probe or the nucleotide primer makes it possible to discriminate between the presence of a first nucleic acid (1) and of a second nucleic acid (2), said nucleic acids (1) and (2) being chosen from the following:
(a) Site −921: the nucleic acid (1) of sequence SEQ ID No. 2 in which the nucleotide at position 41 is a base G and the nucleic acid (2) of sequence SEQ ID No. 2 in which the nucleotide at position 41 is a base A;
(b) Site −438: the nucleic acid (1) of sequence SEQ ID No. 3 in which the nucleotide at position 41 is a base G and the nucleic acid (2) of sequence SEQ ID No. 3 in which the nucleotide at position 41 is a base A;
(c) Site −362: the nucleic acid (1) of sequence SEQ ID No. 4 in which the nucleotide at position 41 is a base A and the nucleic acid (2) of sequence SEQ ID No. 4 in which the nucleotide at position 41 is a base G;
(d) Site −347: the nucleic acid (1) of sequence SEQ ID No. 5 in which the nucleotide at position 41 is a base T and the nucleic acid (2) of sequence SEQ ID No. 5 in which the nucleotide at position 41 is a base C;
(e) Site −296: the nucleic acid (1) of sequence SEQ ID No. 6 in which the nucleotide at position 41 is a base T and the nucleic acid (2) of sequence SEQ ID No. 6 in which the nucleotide at position 41 is a base C;
(f) Site −277: the nucleic acid (1) of sequence SEQ ID No. 7 in which the nucleotide at position 41 is a base T and the nucleic acid (2) of sequence SEQ ID No. 7 in which the nucleotide at position 41 is a base C;
(g) Site −266: the nucleic acid (1) of sequence SEQ ID No. 8 in which the nucleotide at position 41 is a base C and the nucleic acid (2) of sequence SEQ ID No. 8 in which the nucleotide at position 41 is a base T;
(h) Site −168: the nucleic acid (1) of sequence SEQ ID No. 9 in which the nucleotide at position 41 is a base A and the nucleic acid (2) of sequence SEQ ID No. 9 in which the nucleotide at position 41 is a base G;
(i) Site −15: the nucleic acid (1) of sequence SEQ ID No. 10 in which the nucleotide at position 41 is a base A and the nucleic acid (2) of sequence SEQ ID No. 10 in which the nucleotide at position 41 is a base G;
(j) Site +35: the nucleic acid (1) of sequence SEQ ID No. 11 in which the nucleotide at position 41 is a base T and the nucleic acid (2) of sequence SEQ ID No. 11 in which the nucleotide at position 41 is a base C;
(k) Site +515: the nucleic acid (1) of sequence SEQ ID No. 12 in which the nucleotide at position 41 is a base C and the nucleic acid (2) of sequence SEQ ID No. 12 in which the nucleotide at position 41 is a base T;
(l) Site +587: the nucleic acid (1) of sequence SEQ ID No. 13 in which the nucleotide at position 41 is a base C and the nucleic acid (2) of sequence SEQ ID No. 13 in which the nucleotide at position 41 is a base T;
(m) Site +678: the nucleic acid (1) of sequence SEQ ID No. 14 in which the nucleotide at position 41 is a base A and the nucleic acid (2) of sequence SEQ ID No. 14 in which the nucleotide at position 41 is a base G;
(n) Site +960: the nucleic acid (1) of sequence SEQ ID No. 15 in which the nucleotide at position 41 is a base A and the nucleic acid (2) of sequence SEQ ID No. 15 in which the nucleotide at position 41 is a base G;
(o) Site +1059: the nucleic acid (1) of sequence SEQ ID No. 16 in which the nucleotide at position 41 is a base G and the nucleic acid (2) of sequence SEQ ID No. 16 in which the nucleotide at position 41 is a base C;
(p) Site +1068: the nucleic acid (1) of sequence SEQ ID No. 17 in which the nucleotide at position 41 is a base G and the nucleic acid (2) of sequence SEQ ID No. 17 in which the nucleotide at position 41 is a base T;
(q) Site +1473: the nucleic acid (1) of sequence SEQ ID No. 18 in which the nucleotide at position 41 is a base C and the nucleic acid (2) of sequence SEQ ID No. 18 in which the nucleotide at position 41 is a base T;
(r) Site +1867: the nucleic acid (1) of sequence SEQ ID No. 19 in which the nucleotide at position 41 is a base C and the nucleic acid (2) of sequence SEQ ID No. 19 in which the nucleotide at position 41 is a base T;
(s) Site +2939: the nucleic acid (1) of sequence SEQ ID No. 20 in which the nucleotide at position 41 is a base G and the nucleic acid (2) of sequence SEQ ID No. 20 in which the nucleotide at position 41 is a base T;
(t) Site +2983: the nucleic acid (1) of sequence SEQ ID No. 21 in which the nucleotide at position 41 is a base C and the nucleic acid (2) of sequence SEQ ID No. 21 in which the nucleotide at position 41 is a base T;
(u) Site −830 to −824: the nucleic acid (1) of sequence SEQ ID No. 23 and the nucleic acid (2) of sequence SEQ ID No. 22;
(v) Site −580 to −573: the nucleic acid (1) of sequence SEQ ID No. 25 and the nucleic acid (2) of sequence SEQ ID No. 24;
(w) Site +304: the nucleic acid (1) of sequence SEQ ID No. 27 and the nucleic acid (2) of sequence SEQ ID No. 26;
(x) Site +1081: the nucleic acid (1) of sequence SEQ ID No. 29 and the nucleic acid (2) of sequence SEQ ID No. 28;
(y) Site +1505: the nucleic acid (1) of sequence SEQ ID No. 31 and the nucleic acid (2) of sequence SEQ ID No. 30;
(z) Site +1542: the nucleic acid (1) of sequence SEQ ID No. 33 and the nucleic acid (2) of sequence SEQ ID No. 32;
(aa) Site +2514: the nucleic acid (1) of sequence SEQ ID No. 35 and the nucleic acid (2) of sequence SEQ ID No. 34;
(ab) Site +2771: the nucleic acid (1) of sequence SEQ ID No. 37 and the nucleic acid (2) of sequence SEQ ID No. 36; and
(ac) Site +3123: the nucleic acid (1) of sequence SEQ ID No. 39 and the nucleic acid (2) of sequence SEQ ID No. 38.

3. The process as claimed in claim 1, wherein:
a) the nucleotide probe hybridizes specifically with a nucleic acid of a first allelic form of the polymorphic base or of the polymorphic nucleotide sequence defining a first allele of a polymorphic site of the Sh2 gene and does not hybridize with a nucleic acid of a second allelic form of the polymorphic base or of the polymorphic nucleotide sequence defining a second allele of a polymorphic site of the Sh2 gene; or
b) the nucleotide primer hybridizes specifically with a nucleotide sequence contained in an Sh2 gene, said nucleotide sequence being located upstream of an allelic form of a polymorphic base or of a polymorphic nucleotide sequence the presence or absence of which defines an allele of a polymorphic site of the Sh2 gene.

4. The process as claimed in claim 1, wherein the improved phenotypic seed quality characteristics are chosen from the number of seeds per ear, the seed mass, the protein content of the seeds, the starch content of the seeds, the amylose content of the seeds and the protein/starch weight ratio in the seeds, or a combination of these phenotypic characteristics.

5. A process for determining the identity of the allele of a polymorphic site within a nucleic acid derived from an Sh2 gene for the purpose of selecting a plant having improved phenotypic seed quality characteristics, characterized in that it comprises a step consisting of characterizing the identity of the polymorphic base or of the polymorphic nucleotide sequence present at at least one nucleotide position of said nucleic acid corresponding to at least one of the nucleotides at position −921, −830 to −824, −580 to −573, −438, −362, −347, −296, −277, −266, −168, −15, +35, +304, +515, +587, +678, +960, +1059, +1068, +1081, +1473, +1505, +1542, +1867, +2514, +2771, +2939, +2983 and +3123 of the Sh2 gene of sequence SEQ ID No. 1.

6. The process as claimed in claim 5, which comprises carrying out the characterization of the identity of the polymorphic site by sequencing said nucleic acid.

7. The process as claimed in claim 5, characterized in that the characterization of the identity of the polymorphic site is carried out by hybridization of a nucleotide probe which hybridizes specifically with a polymorphic base or with a polymorphic nucleotide sequence defining an allele of a given polymorphic site of the Sh2 gene.

8. The process as claimed in claim 5, which comprises carrying out the characterization of the polymorphic site by extending a nucleotide primer which hybridizes specifically with a nucleotide sequence located upstream of a polymorphic base or of a polymorphic nucleotide sequence defining an allele of a given polymorphic site of an Sh2 gene.

9. The process as claimed in claim 5, which comprises, in order to select a plant having a modified number of seeds, determining the identity of the base or of a sequence of bases present at at least one nucleotide position of said nucleic acid corresponding to at least one of the nucleotides at position −168, +1473, +1542 and +2983 of the Sh2 gene of sequence SEQ ID No. 1.

10. The process as claimed in claim 5, which comprises, in order to select a plant with a modified seed mass, determining the identity of the base or of a sequence of bases present at at least one nucleotide position of said nucleic acid corresponding to at least one of the nucleotides at position −168, +1473, +1542 and +2983 of the Sh2 gene of sequence SEQ ID No. 1.

11. The process as claimed in claim 5, which comprises, in order to select a plant having a modified protein content in the seed, determining the identity of the base or of a sequence of bases present at at least one nucleotide position of said nucleic acid corresponding to at least one of the nucleotides at position −168, +1473, +1542, +2983, −830 to −824, −362, −347, −296, −15, +515, +587, +1068, +1505 and +2939 of the Sh2 gene of sequence SEQ ID No. 1.

12. The process as claimed in claim 5, which comprises, in order to select a plant having a modified starch content in the seed, determining the identity of the base or of a sequence of bases present at at least one nucleotide position of said nucleic acid corresponding to at least one of the nucleotides at position −830 to −824, −362, −347, −296, −15, +515, +587, +1068, +1505 and +2939 of the Sh2 gene of sequence SEQ ID No. 1.

13. The process as claimed in claim 5, which comprises, in order to select a plant having a modified amylose content in the seeds, determining the identity of the base or of a sequence of bases present at at least one nucleotide position of said nucleic acid corresponding to at least one of the nucleotides at position −438, −266, +678, +960, −921, −580 to −573, −277, +35, +304, +1059, +1081, +1867, +2514, +2771 and +3123 of the Sh2 gene of sequence SEQ ID No. 1.

14. The process as claimed in claim 5, which comprises, in order to select a plant having a modified protein/starch ratio in the seed, determining the identity of the base or of a sequence of bases present at at least one nucleotide position of said nucleic acid corresponding to at least one of the nucleotides at position −168, +1473, +1542, +2983, −830 to −824, −362, −347, −296, −15, +515, +587, +1068, +1505 and +2939 of the Sh2 gene of sequence SEQ ID No. 1.

15. The process as claimed in claim 5, which is carried out on the DNA taken from plants at the plantlet stage and/or at the early stage and/or at the vegetative stage.

16. The process as claimed in claim 5, wherein the plant is a cereal.

17. The process as claimed in claim 16, wherein the plant is maize or sorghum.

18. A nucleotide probe or a nucleotide primer, characterized in that it makes it possible to distinguish between the various alleles of a polymorphic site at at least one of the positions −921, −830 to −824, −580 to −573, −438, −362, −347, −296, −277, −266, −168, −15, +35, +304, +515, +587, +678, +960, +1059, +1068, +1081, +1473, +1505, +1542, +1867, +2514, +2771, +2939, +2983 and +3123 of the Sh2 gene of sequence SEQ ID No. 1.

19. The use process which comprises marking at least one polymorphic site of the Sh2 gene with a probe or a primer as claimed in claim 18.

20. A diagnostic set or kit to predict the phenotypic plant seed quality characteristics, which comprises:
a) a probe or a plurality of probes or primers as claimed in claim 18; and
b) where appropriate, reagents required to carry out a hybridization or amplification reaction.

21. A nucleic acid as claimed in claim 39 capable of conferring on a plant a modified number of seeds compared to the reference “wild-type” maize, wherein said nucleic acid comprises the allelic form associated with the expression of the modified phenotypic seed quality characteristic at at least one polymorphic site chosen from the polymorphic sites −168, +1473, +1542 and +2983 of the Sh2 gene of sequence SEQ ID No. 1.

22. A nucleic acid as claimed in claim 39 capable of conferring on a plant a modified seed mass compared to the reference “wild-type” maize, wherein said nucleic acid comprises the allelic form associated with the expression of the modified phenotypic seed quality characteristic at at least one polymorphic site chosen from the polymorphic sites −168, +1473, +1542 and +2983 of the Sh2 gene of sequence SEQ ID No. 1.

23. A nucleic acid as claimed in claim 39 capable of conferring on a plant a modified protein content in the seeds compared to the reference “wild-type” maize, wherein said nucleic acid comprises the allelic form associated with the expression of the modified phenotypic seed quality characteristic at at least one polymorphic site chosen from the polymorphic sites −168, +1473, +1542, +2983, −830 to −824, −362, −347, −296, −15, +515, +1068, +1505 and +2939 of the Sh2 gene of sequence SEQ ID No. 1.

24. A nucleic acid as claimed in claim 39 capable of conferring on a plant a modified starch content in the seeds compared to the reference “wild-type” maize, wherein said nucleic acid comprises the allelic form associated with the expression of the modified phenotypic seed quality characteristic, at at least one polymorphic site chosen from the polymorphic sites −830 to −824, −362, −347, −296, −15, +515, +587, +1068, +1505 and +2939 of the Sh2 gene of sequence SEQ ID No. 1.

25. A nucleic acid as claimed in claim 39 capable of conferring on a plant a modified amylose content in the seeds compared to the reference “wild-type” maize, wherein said nucleic acid comprises the allelic form associated with the expression of the modified phenotypic seed quality characteristic at at least one polymorphic site chosen from the polymorphic sites −438, −266, +678, +960, −921, −580 to −573, −277, +35, +304, +1059, +1081, +1867, +2514, +2771 and +3123 of the Sh2 gene of sequence SEQ ID No. 1.

26. A nucleic acid as claimed in claim 39 capable of conferring on a plant a modified protein/starch ratio in the seed compared to the reference “wild-type” maize, wherein said nucleic acid comprises the allelic form associated with the expression of the modified phenotypic seed quality characteristic at at least one polymorphic site chosen from the polymorphic sites −168, +1473, +1542, +2983, −830 to −824, −362, −347, −296, −15, +515, +587, +1068, +1505 and +2939 of the Sh2 gene of sequence SEQ ID No. 1.

27. A recombinant vector comprising a nucleic acid as claimed in claim 39.

28. A method which comprises transforming a host cell with a nucleic acid or with a recombinant vector comprising a nucleic acid, and wherein the nucleic acid is a nucleic acid as claimed in claim 39.

29. A method as claimed in claim 28, wherein the host cell is a bacterial host cell or plant host cell.

30. A host cell transformed with a nucleic acid or with a recombinant vector comprising a nucleic acid, wherein the nucleic acid as claimed in claim 39.

31. The transformed host cell as claimed in claim 30, which is a bacterial cell or a plant cell.

32. A method which comprises producing seeds with improved industrial or agrofoods qualities with a) a nucleic acid, b) a recombinant vector comprising a nucleic acid, c) a host cell transformed with a nucleic acid, or d) a host cell transformed with a recombinant vector comprising a nucleic acid, wherein the nucleic acid is a nucleic acid as claimed in claim 39, and the host cell is, optionally, a bacterial cell or a plant cell.

33. A transformed plant comprising a plurality of host cells as claimed in claim 30.

34. A process for obtaining a transformed plant capable of producing seeds with improved industrial or agrofoods qualities, which comprises the following steps:
a) transforming at least one plant cell with a nucleic acid or with a recombinant vector comprising a nucleic acid, wherein the nucleic acid is a nucleic acid as claimed in claim 39;
b) selecting the transformed cells obtained in step a) which have integrated into their genome at least one copy of a nucleic acid as claimed in claim 39; and
c) regenerating a transformed plant from the transformed cells obtained in step b).

35. A transformed plant or a part of a transformed plant which can be obtained by the process as claimed in claim 34.

36. A product of transformation of a grain or seed as claimed in claim 35.

37. An antibody specific for an SH2 polypeptide encoded by a nucleic acid as claimed in claim 39.

38. A pack or kit for diagnosing phenotypic plant seed quality characteristics, which comprises:
a) an antibody or a combination of antibodies as claimed in claim 37;
b) where appropriate, the reagents required for the detection of a complex formed between said antibody or antibodies and an SH2 polypeptide.

39. A nucleic acid capable of conferring on a plant:
a) a modified number of seeds compared to a reference “wild-type” maize,
b) a modified seed mass compared to a reference “wild-type” maize,
c) a modified protein content in the seeds compared to a reference “wild-type” maize,
d) a modified starch content in the seeds compared to a reference “wild-type” maize,
e) a modified amylose content in the seeds compared to a reference “wild-type” maize, or
f) a modified protein/starch ratio in the seed compared to a reference “wild-type” maize;
wherein said nucleic acid comprises the allelic form associated with the expression of the modified phenotype seed quality characteristic,
for each of a) and b), as defined in the present description, at at least one polymorphic site chosen from the polymorphic sites −168, +1473, +1542 and +2983 of the Sh2 gene of sequence SEQ ID No. 1;
for c), as defined in the present description, at at least one polymorphic site chosen from the polymorphic sites −168, +1473, +1542, +2983, −830 to −824, −362, −347, −296, −15, +515, +587, +1068, +1505 and +2939 of the Sh2 gene of sequence SEQ ID No. 1;
for d), as defined in claim 1, at at least one polymorphic site chosen from the polymorphic sites −830 to −824, −362, −347, −296, −15, +515, +587, +1068, +1505 and +2939 of the Sh2 gene of sequence SEQ ID No. 1;
for e), as defined in claim 1, at at least one polymorphic site chosen from the polymorphic sites −438, −266, +678, +960, −921, −580 to −573, −277, +35, +304, +1059, +1081, +1867, +2514, +2771 and +3123 of the Sh2 gene of sequence SEQ ID No. 1; and
for f), as defined in claim 1, at at least one polymorphic site chosen from the polymorphic sites −168, +1473, +1542, +2983, −830 to −824, −362, −347, −296, −15, +515, +587, +1068, +1505, and +2939 of the Sh2 gene of sequence SEQ ID No. 1.
